(19) United States
(12) Patent Application Publication
(10) Pub. No.: US 2004/0037780 A1
(43) Pub. Date: Feb. 26, 2004

(54) Respiratory delivery for gene therapy and lentiviral delivery particle

(76) Inventors: David Parsons, Marino (AU); Don Anson, Thebarton (AU); Maria Limberis, Rostrevor (AU); Maria Fuller, Prospect (AU)

Correspondence Address: Henry D. Coleman, Coleman Sudol Sapone, P.C., 714 Colorado Avenue, Bridgeport, CT, 06605-1601, US

(21) Appl. No.: 10/226,638

(22) Filed: Aug. 23, 2002

(30) Foreign Application Priority Data
Nov. 19, 2001 (AU) PR8942

(51) Int. Cl. A61K 48/00; A61L 9/04

(52) U.S. Cl. 424/45; 514/44

(57) ABSTRACT

Delivery of an exogenous gene to the respiratory epithelium in a lentiviral expression vector. Includes the preconditioning of the respiratory tract with a penetrating agent such as a detergent to cause tolerable transient damage to the superficial epithelial cell layer. The effective amount of the penetrating agent may be determined by measuring a drop of TPD. Utilising LPC the present inventors have found persistence of expression of an exogenous gene for periods exceed the turnover time of epithelial cells. Additionally amelioration of the pulmonary manifestation of Cystic Fibrosis has been monitored in a mouse modely using th epresent invention. Additionally a safe lentiviral particle packaging system is described where the Gag protei and the GagPol proteins are separately expressed with mutation of the frameshift site in nucleic acid encoding the GagPol protein ensuring that both proteins cannot expressed.

### FIELD OF THE INVENTION

This invention relates to a method for respiratory delivery for gene therapy purposes and the treatment of a condition that has respiratory or pulmonary manifestations and a vector and method for treatment or prevention of the condition. Specific aspects of the invention relate to treatment of cystic fibrosis by gene therapy using a lentiviral particle to introduce the CTFR gene into cells of the lung with expression of CTFR that is persistent. Further specific aspects relate to a lentiviral based delivery system useful for gene therapy and therapeutic agent delivery applications.

### BACKGROUND OF THE INVENTION

At present, a primary limitation in the use of gene therapy to treat human disease is the ineffectiveness of gene delivery methods.

Respiratory delivery encounters a more complex defence system than that confronted by other delivery mechanisms. The highly evolved effective defences protecting the mammalian airway epithelium against allergens, irritants, dust, viruses and microbial pathogens (Bevans, 1999) also apply to gene transfer vectors. In particular, the superficial airway mucus layer, produced by submucosal glands and goblet cells continually captures inhaled or introduced particles for constant removal by mucociliary clearance. Closer to the cell surface the glycocalyx (Pickles et al., 2000) can bind some vector types, further preventing vector particle entry via the apical cell membrane. Finally, the tight-junctions between epithelial cells form yet another major delivery barrier to those gene transfer vectors that can bind to their specific receptors only abundant on basolateral cell membranes (Bergelson et al, 1997).

A large number of proposals have been made in the delivery of medications via the lung, many of these attempt to use the large mucosal surface area of the lung to absorb pharmaceuticals such as insulin to the systemic system. There are continuing difficulties being addressed for such systems, however these systems, whilst they address the minimisation of damage to cells of the lung, do not require uptake and integration of nucleic acid.

Attempts at providing nucleic acids for incorporation into cells lining the respiratory system are ongoing, and generally utilise a viral delivery vector, or nucleic acid packaged in a non viral delivery vehicle such as cationic lipid systems. A difficulty encountered in these attempts has been the issue of the persistence of expression of the nucleic acid. A major concern regarding the use of viruses is that of safety and for that reason non-viral delivery systems have been regarded by some as perhaps having the best prospect for resulting in a commercially acceptable product.

Cystic Fibrosis (CF) is the most common, life-threatening, autosomal recessive disease, in the Caucasian population. Though many organs are affected by the ion imbalances induced by a malfunctioning cell chloride channel the cystic fibrosis transmembrane conductance regulator (CFTR)-, it is the chronic and progressive infective lung disease that produces the high levels of mortality and morbidity experienced by CF children and adults. Recent studies suggest that the primary effect in the CF airway of the derangement of cell ion balance is a reduced airway surface liquid (ASL) volume (Tarran et al., 2001). Lowered ASL volume prevents normal operation of cell cilia, with ineffective mucociliary clearance causing mucus stasis, allowing inhaled bacteria to deposit, proliferate, and initiate chronic lung disease.

The discovery of the CFTR gene in 1989 triggered research into the use of gene therapy (Mulligan, 1993) to produce a cure for CF. Gene therapy depends on the delivery of a functional copy of CFTR into the affected cells of the conducting airways to restore correct cell function (which may be measured as a return of normal surface electrophysiology) and thus prevent CF disease progression. While the theoretical basis of gene therapy for CF is conceptually simple, and is easily demonstrated in vitro (Johnson et al., 1992), many technical barriers to its application in vivo have been identified during Phase I clinical trials (Albelda et al, 2000, Davies et al., 2001) of the delivery of the CFTR gene to nasal airway using adenovirus, adeno-associated virus or cationic lipid vectors. One of these technical barriers is overcoming the additional barrier for delivery encountered in CF patients that result from secondary infection and lack of clearance of the lungs.

Modulating the effectiveness of these apical surface airway barriers, for example by opening epithelial tight junctions for virion access to receptors, is an approach to improving in vivo gene transfer that has recently received attention. In particular, airway treatment with EGTA (Wand et al, 2000, Chu et al, 2001), a synthetic detergent (Parsons et al, 1998), or a detergent component of pulmonary surfactant (Parsons et al., 1999), can improve in vivo gene transfer to airway cells in animal models.

The identity of the specific cells in the airway epithelium that express CFTR cannot be accurately determined by immunocytochemical means, because of the low abundance of protein. However, functional studies suggest that the ciliated epithelial cells and perhaps nonciliated cells of the surface epithelium are among the main cell types involved in ion balance. Thus, in practical terms, the present preferred target cell for gene therapy would appear to be the mature cells that line the pulmonary airways. These are not rapidly dividing cells; rather, most of them are nonproliferating and many may be terminally differentiated.

Lentiviruses are a subgroup of retroviruses that are capable of infecting non-dividing as well as dividing cells and this has been considered by the inventors as a promising prospect for delivery of CFTR should the serious safety concerns due to the possibility of recombination by the vector into a virulent and disease-causing form be addressed, and of course the persistence of expression of the integrated gene be demonstrated in sufficient quantities to be useful.

Although lentivirus vectors can transduce dividing and non-dividing cells, provide stable and sustained gene expression, and evade host inflammatory and immune responses (Amado et al., 1999)—all desirable features of airway gene therapy—this has not been demonstrated to date in vivo (West & Rodman, February 2001).

U.S. Pat. No. 5,641,662 by Debs et al., demonstrate the delivery of DNA packaged in a non-viral system to cells of the epithelial cells and demonstrates expression of integrated cells. Debs et al., however have not shown that the effect has an overall physiological benefit that is persistent.

U.S. Pat. No. 6,093,567 to Gregory, et al. discloses the use of adenovirus in a gene therapy approach to cystic fibrosis, using no penetration enhancer. The effects are however transitory, and are recognised by the authors as such.

### SUMMARY OF THE INVENTION

A first major aspect of this invention arises from the finding that the delivery of a recombinant lentivirus carrying an exogenous gene to the respiratory system, following the first delivery of a non-toxic amount of a penetration enhancer can provide persistence of expression of a gene product. In one form the finding has been in relation to the delivery of the gene encoding CFTR to epithelial cells. This has particular implications in alleviating the manifestations of Cystic Fibrosis associated with the lungs.

This is the first time to the inventor's knowledge that a viral based expression system used for in vivo respiratory tissue delivery has resulted in persistent in situ expression of an exogenous gene. It is also, to the inventors belief, the first time that the electrochemical imbalance cause by cystic fibrosis has been shown to be reduced for an extended period by the delivery of a single dose to the respiratory system of nucleic acid encoding CFTR.

Without being bound by these explanations one reason for the result achieved by the inventions might be that the treatment of the lung by a penetration enhancer is at a time and in quantities that cause sufficient but transient tolerable damage of cell layer integrity of cells lining the pulmonary system to at least make permeable the tight junctions that act as a barrier to ingress of exogenous material, to thereby enable chromosomal integration of the exogenous nucleic acid relatively rapidly. It is additionally speculated that perhaps the transient damage may lead to stimulation of progenitor cell acitiviy to enhance the capacity of persistence of gene expression in epithelial cells

It is believed that the choice of a lentiviral delivery treatment means that the levels of nucleic acid to be provided to the lung can be kept lower than other means of packaging recombinant exogenous genes and that the choice of vector type together with the pretreatment makes possible not only infection, but more importantly places sufficient numbers of the cells in a state that is able to integrate the exogenous gene and lead to persistence. Additionally whilst the target cells have been suspected to be mature cells lining the lung airways this has never specifically been demonstrated before at any level of confidence in regard to succesful CFTR gene delivery.

This finding of the persistence of expression of the delivered gene is very likely to have application to the treatment of conditions other than cystic fibrosis and the invention may be applicable to a range of other conditions with the delivery of other genes.

In a first broad aspect the invention might be said to reside in a method of delivering one or more exogenous genes for expression in an epithelial cell in the respiratory system of a mammal to give persistent expression of the gene in the epithelial cell, the method including the steps of delivering an effective amount of a penetrating agent to cause tolerable transient damage to the integrity of the superficial epithelial cell layer of the respiratory system, and the step of delivering a recombinant exogenous gene in a lentiviral particle, the lentiviral particle containing a non-replicating nucleic acid, the nucleic acid encoding the exogenous gene operably linked to a control sequence for controlling expression of the gene.

In a second form of the first broad aspect the invention might be said to reside in a method of delivering one or more exogenous genes for expression in an epithelial cell in the respiratory system of a mammal to give persistent expression of the gene in the epithelial cell, the method including the steps of delivering an effective but non-damaging amount of a penetrating agent to the respiratory system, and the steps of delivering a recombinant exogenous gene in a lentiviral particle in an effective dose to the respiratory system, for integration into a chromosome of sufficient cells in the pulmonary system to provide an ameliorating effect for a condition, the lentiviral particle containing a non-replicating nucleic acid, the nucleic acid encoding the exogenous gene operably linked to a control sequence, for controlling expression of the gene.

The first broad aspect of the invention might also encompass a method of preventing or treating the respiratory manifestations of cystic fibrosis in a mammal. The administration of the above method is preferably before severe respiratory manifestations arise to thereby prevent non-reversable damage that occurs, however the quality of life of cystic fibrosis sufferers with milder airway disease may be improved by treatment by this invention. The first broad form of this invention might also encompass formulations of penetration enhancers with a recombinant lentivirus carrying the CFTR gene.

The inventors have also shown that the Gag and GagPol polyproteins can be efficiently expressed at high levels from separate expression constructs allowing the removal of the HIV-1 gagpol translational frameshift sequence from the virus production system. This separation of Gag and GagPol and the reduction or minimisation of the frameshift event provides the basis for the production of recombinant HIV-1 vectors useful for gene therapy applications. In the illustrated embodiment the improved safety of this approach has been demonstrated by the incidence of transfer of biologically active sequences encoding Gag/GagPol to transduced cells.

Thus in a broad second aspect the invention might be said to reside in a recombinant lentiviral packaging system. A replication deficient lentivirus vector can be replicated and packaged into a lentiviral particle in a lentivirus permissive cell in the presence of both a Gag encoding nucleic acid sequence and GagPol encoding nucleic acid sequence where the Gag and GagPol are encoded by different coding sequences on separate expression constructs. Thus while any one viral particle of the deficient lentivirus so produced might encode the Gag or the GagPol polyprotein via recombination with one of said expression plasmids but not both and a second viral particle may encode the other, coincident encoding of both in one viral particle is prevented, more preferably the replication deficient lentivirus will not encode either. Other proteins required for packaging and replication will be supplied by other expression constructs. The GagPol encoding sequence is a modified sequence that no longer encodes for the full Gag protein, and has modified the nucleic acid sequence of the frameshift site such that the frameshift no longer occurs. The modified sequence alters the seven nucleotide HIV-1 frameshift sequence (AUUUUUU) (Jacks, T., Power, M D., Masiarz, P A., Luciw, P A., Barr, P J., Varmus, H E. 1988. Characerisation of ribosomal framshifting in HIV-1 gag-pol expression. Nature 331, 280-283) to the non-functional sequence ACUUCCU. Any other sequence with conservative changes that no longer functions as a frameshift signal could also be used. Preferably the GagPol encoding sequence also has further conservative modifications to nucleotides associated with the hairpin structure associated with the frameshift side are modified such that the hairpin structure is less stable or does not form at all. It will be understood that the Gag protein is encoded by a nucleic acid sequence that is a truncation in relation to the normal GagPol protein, and can therefore no longer form GagPol.

The invention also encompasses vectors useful for the replication and packaging of this second broad aspect of the invention as well as viral particles formed using the packaging system.

Both Gag and GagPol are essential for the replication a lentivirus and in the absence of either one the replication/deficient vector will not infect other cells. The net effect of the modifications to the sequence encoding GagPol and ensuring that both are not present on the replication/deficient lentivirus is that this becomes acceptably safe and might be used as a vector for delivery for gene therapy purposes.

It will also be understood that the second broad aspect of the invention also encompasses a GagPol encoding nucleic acid that no longer encodes for the full Gag protein, and has modified the nucleic acid sequence of the frameshift site. Preferably the GagPol encoding sequence also has further conservative modifications to nucleotides associated with the hairpin structure associated with the frameshift side are modified such that the hairpin structure is less stable or does not form at all. The modified GagPol sequence has been deposited in GenBank Accession number AF287353 and is shown in FIG. 18.

### BRIEF DESCRIPTION OF THE DRAWINGS

FIG. 1 LV vector constructs (i) The LVLacZa vector contains from 5′ to 3′ the HIV-1 YU-2 5′ viral long terminal repeat (LTR) and contiguous sequence extending 1150 base pairs into the gag gene, the cPPT sequence, the SV40 immediate early promoter, the LacZ gene sequence, the HIV-1 YU-2 polypurine tract (PPT) and the 25 base pairs sequence immediately 5′ of the PPT, and the 3′ LTR with the rev response element (RRE) replacing sequences between the EcoRV and PvuII sites in the U3 region. This strategy renders the vector self-inactive as vital transcriptional elements in U3 region have been replaced by the RRE sequence. (ii) In the LVLacZb vector the length of the gag sequence is reduced to 550 base pairs and an extended RRE sequence is positioned immediately 5′ of the cPPT rather than in the 3′ LTR. The construct was made self-inactivating by deleting the sequences between the EcoRV and PvuII sites in the 3′ LTR (ΔU3), and (iii) The LVCFTR vector construct is similar to the LVLacZb vector construct described above with the difference that the gag reading frame was blocked by mutagenesis of the ATG codons at base 788 and 1298 (HIV-1 YU-2, GenBank accession number M93258) of the HIV-1 YU-2 sequence to TAG stop codons and the CFTR cDNA sequence replaces the LacZ marker gene sequence.

FIG. 2 LacZ gene transfer after a single delivery of LVLacZ into the LPC pre-treated right mouse nasal airway. (A): 7 days. En face anteriorly-directed view of septum (s) and turbinates (nasoturbinate (nt), maxilloturbinate (mt)) at 7 days. Undosed (left) nasal airway displays no LacZ positive cells, or regions, while the treated (right) side shows scattered LacZ positive cells along the vertical face of the septum and the some faces of the nasal turbinates. Thick arrow shows direction of view of septum face in panels C and E. Note lack of LacZ positive cells in the untreated nasal airway. Section is similar to that of Level 16 (Mery et al., 1994), where extensive detail on mouse nasal airway anatomy and cell types is available. (B): 7 days. Situated below the brain (br) at the posterior of the nasal cavity, moderate cell staining is present in the nasopharyngeal meatus on only the ipsilateral (right) portion of this airway, corresponding to the dosed nostril (arrow). (C): 28 days. View of the septum wall as indicated by arrow in (A). The remaining nasal airway of the ispilateral (dosed) nostril has been cut away to allow this view. The patchy punctate blue staining of LacZ positive cells that contrasts with the diffuse blue-green background stain characteristic of X-gal processing in un-sectioned tissue (here, in the olfactory region) is apparent. (D) 28 days. LacZ positive cells in nasopharyngeal meatus (arrow) again remain ipsilateral. (E): 92 days. View of the X-gal processed septum and left nostril (the curve of the contralateral dorsal olfactory region is visible here outlined by the diffuse background X-gal stain. Scale bar applies to A, C, E. (F): 92 days. Strong and extensive LacZ positive cell present in the ipsilateral half of the nasopharyngeal meatus (arrow) reveals persistence of LacZ expression for 3 months after the single dose of the LV vector. Scale bar applies to B, D, F.

FIG. 3. Details of anterior nasal LacZ gene expression after LV-mediated gene transfer. LacZ gene transfer into ciliated airway epithelium on the nasal septum was limited to the right treated nostril. Saf-O stained sections at (a) 7 days, (b) 28 days, and (c) 92 days after exposure to the LVLacZ vector show gene expression as individual blue darkly-stained cells, or groups of LacZ positive cells (arrows).

FIG. 4. Airway LacZ gene expression in nasal airway. (a): Only when the LVLacZ vector instillation was combined with LPC pre-treatment (or PDOC pre-treatment, not shown) was LacZ gene transduction observed. The combination of 1% LPC pre-treatment and polybrene-free LVLacZa vector preparation resulted in the greatest gene transfer, shown here 7 days after dosing (*P<0.05, ANOVA, n=3 per group). (b): Quantitation of LacZ gene expression over three standard nasal airway cross sections supports the qualitative impression (FIG. 2) of persistence of LacZ gene expression for at least 92 days after dosing. The apparent increase in transduction at 92 days was not significant (P=0.64), though statistical power was low (power=0.05, ANOVA, n=3 per group).

FIG. 5. Types of LacZ positive cells in mouse nasal airway. The significance of changes in the proportion of transduced cells of each cell type over the three assessment time points was individually examined using logistic regression analysis. For each cell type the proportion of transduced cells altered significantly during the assessment period (*Ciliated P=0.01, Non-ciliated P<0.001, Secretory P<0.001, Basal P<0.001). Statistically, the significance of the results for the Secretory cells should be regarded as approximate, given the zero counts on days 7 and 28.

FIG. 6. Effect of LVCFTR administration on nasal airway ΔPD. (a): Between 7 days and 13 months the mean change in ΔPD (horizontal bars: mean with SE) as well as individual (time-linked) ΔPD values (symbols ▪, ♦, ▾, ▴, , ●) are shown. Significant partial correction of CFTR electrophysiological function compared to the mean ΔPD of untreated CF mice (“CF”, n=6) was present at 46 days (*P<0.05, ANOVA, Dunnett's multiple comparison, n=3). At 110 days the ΔPD of one mouse remained high, but by 13 months the ΔPD for both remaining mice in this study had waned to near untreated CF mouse ΔPD mean values. (b): Basal TPD of nasal airway following LVCFTR dosing protocol. ANOVA analysis indicated that there was a significant difference between the five treatment groups (P=0.03) but subsequent multiple comparisons against the untreated CF control group (Dunnett's method) did not identify the source of the significant TPD reduction(s). Power (0.58) was below that required in this study (0.80).

FIG. 7 Is a histogram showing the efficacy of recombinant adenoviral expression of genes into nasal epithelial cells after 3 days following infection with pretreatment with 10 μl of the detergent 1 hour prior to administration of the recombinant adenovirus.

FIG. 8 Is a histogram showing the effect of treatment with 4 μl of PBS, PDOC or LPC at four different concentrations on TPD relative to pretreatment (taken as a value of 100%).

FIG. 9 Is a histogram showing the effect of treatment with 4 μl of LPC at three different concentrations over time on TPD relative to pretreatment (taken as a value of 100%).

FIG. 10 Is a histogram showing the effect of treatment with 4 μl of PDOC at three different concentrations over time on TPD relative to pretreatment (taken as a value of 100%).

FIG. 11 Is a histogram showing the effect of treatment with 4 μl of PDOC at three different doses over time on TPD relative to pretreatment.

FIG. 12. Vector constructs. pB1HIVext5SV40EYFPppt+RRELTR (Fuller and Anson, unpublished) contains, from 5′ to 3′, 5′ HIV-1 YU-2 long terminal repeat (U3/R/U5) and contiguous packaging signal (ψ), splice donor site (SD) and 1150 bp of 5′ gag gene sequences, the SV40 early promoter (SV40), enhanced yellow fluorescent protein coding sequence (EYFP), HIV-1 YU-2 polypurine tract and 3′ long terminal repeat containing the HIV-1 YU-2 RRE sequence (RRE) cloned between the EcoRV and PvuII sites in the U3 region (replacing the U3 sequence between these sites). This vector is therefore self-inactivating. pB1HIVext5SV40Neoppt+RRELTR is an analogous construct transducing Neo^{r} rather than EYFP.

FIG. 13. Expression constructs for HIV-1 Gag/GagPol/Pol. The figure shows the basic expression constructs for the gagpolml, gagml, gagpolfusionml and polml codon-optimised sequences. DNA coding sequences are shown as solidly shaded boxes (black for gag, grey for pol) and the corresponding polyproteins are indicated by stippled boxes (Gag by dots, Pol by chevrons). The CMV promoter is indicated by the horizontal triangle and the bovine growth hormone polyadenylation sequence by “poly(A)”. The frameshift signal in the gagpolml sequence is indicated by “−1 fs”.

FIG. 14. Western blot analysis of p24 expression. The stated plasmids were transfected into 293T cells and 72 hours later cell lysates prepared and analysed by western blot analysis using antiserum from a HIV-1 positive individual as described in Materials and methods. Lane 1, control (mock transfected) cells; lane 2, pCMVΔRnr transfected cells; lane 3, pcDNA3gagpolml transfected cells; lane 4, pcDNA3gagml plus pcDNA3gagpolfusionml transfected cells. The sizes of the molecular weight standards are indicated to the left of the figure. An arrow to the right of the figure indicates the position of p24.

FIG. 15. Western blot analysis of Vpr expression. pcDNA3Vpr was transfected into Cos-1 cells and 72 hours later cell lysates prepared and analysed by western blot analysis using antiserum to Vpr (NIH AIDS research and reference reagent program cat. no. 3252) as described in Materials and methods. Lane A, lysate from pcDNA3Vpr transfected cells; lane B, lysate prepared from control cells. Molecular weight standards are indicated to the left of the figure. The arrow on the right indicates the position of the protein in transfected, but not control, lysates reacting with the antiserum.

FIG. 16 Shows the DNA sequence of the modified GagPol nucleic acid sequence, that no longer exhibits the high freqeuncy frameshift in translation exhibited by the wild type.

FIG. 17 Shows the hairpin structures and the frame shift site associated with the GagPol sequence in the wild type sequence and the sequence as modified for this invention.

### DETAILED DESCRIPTION OF THE INVENTION

Turning to the first broad aspect of this invention. The endpoint of the persistence of expression of the exogenous gene has not been determined. It is hoped that this approach will result in amelioration that will extend much further than the few months that the experimental data shown herein has demonstrated, however it is believed that a persistence of greater than 1 month will provide for beneficial effect although more preferably that will be greater than 2 months or 3 months. It is believed that the cells that have the effect of ameliorating the respiratory manifestations of cystic fibrosis are terminally differentiated so that even though it may be desirable to have stem cells or other progenitor cells infected there is a prospect that these are not infected or insufficient numbers of the progenitors are infected such that periodic boosting of the treatment may be required. The terminally differentiated cells are unlikely to divide and after a time it is anticipated that there will be apoptosis of the cells and/or their progeny.

In one form the product of the one or more exogenous genes is one that is transported apically or has an affect in the air passageway exposed to the lung or pulmonary system and thus might address a condition that has symptoms manifest as a result of a protein or other product failing to perform its normal fucntion via the apical surface of epithelial cells. This fits in with the generally held view supported by work done on the well studied proteins sysnthesis by the polarised airway epithelium. The exogenous gene might encode a protein that addresses a defect in the cell in which the exogenous nucleic acid integrates and perhaps adjacent cells. In a very specific form of this invention the exogenous gene encodes for CFTR. Another deficiency with respiratory manifestations that might also be treated in like manner is alpha-1 antitrypsin deficiency

There is work however that shows that protein products can be secreted from tehlung to the blood. For a protein product to be delivered from tehlung epithelia to the blood it must be secreted from teh basolateral membrane. recent work has shown that that mode of delivery does occur for facto IX, Epo and AAT (Auricchio et al., 2002, Siegfried et al., 1995). The present invention also encompasses that the product of the one or more exogenous genes may be used for that purpose and might therefore include a secretory string of amino acids to facilitate export through the basolateral membrane.

The nucleic acid of the lentivirus is non-replicating. Such delivery particles are well known. Several retroviral delivery systems have been described in the past. These generally involve a system for packaging made from two or more plasmids that encode proteins essential for the packaging of modified viral nucleic acid, and for replication of the modified viral nucleic acid. The modified viral nucleic acid encodes the exogenous gene and operably linked control regions, as well as genes and nucleic acid string essential for packaging, and for integration into a chromosome of the host cell, however does not encode genes essential for replication This therefore represents a non-replicating viral particle.

The control region operably linked to the exogenous gene or genes will most preferably encode a promoter adjacent the exogenous gene which enhances the expression of the exogenous gene. Although it might be sufficient to rely on endogenous promoters nearby the integrated nucleic acid to provide expression that is not preferred. Other control elements might make the expression specific for the cells to be targeted. Thus, for example, one or more enhancers might be provided to specifically enhance transduction and expression in the cells to be targeted. Additionally other control elements might also be included such as cell specific repressor sites that for instance repress expression in cell types where expression might be detrimental.

It might also be desired to include a termination signal after the exogenous gene to minimise disruption of the expression of any endogenous genes adjacent the integration site. Such termination signals do however tend to be universal and accordingly any termination signal might be sought.

Viral vectors are particularly efficient at delivery of DNA to cells. Viral particles are generally resilient to a range of defence mechanisms present in the lung, additionally retroviruses have quite effective mechanisms for integration of their nucleic acids into the chromosome of a host. Thus generally less viral particles are required to effectively lead to an integration event than nucleic acid delivered in other ways. Thus the choice of a viral particle has the potential for providing an efficient means of infecting cells of the pulmonary system compared to the use of other DNA delivery mechanism. This is a pertinent consideration because it is known that the delivery by other mechanism requires that such larger amounts must be delivered so as to cause adverse reactions by the recipient.

Many viruses are, however, limited from a point of view of the types of cells that are infected and the state of cells with regard to the cell cycle. Integration of nucleic acid in to a host cell for many retroviruses tend to require cell division. It has been postulated that cells that are appropriate targets, for example for CFTR, are terminally differentiated cells and accordingly the viral particle must be selected to infect non-dividing cells. It is generally recognised that lentiviruses are able to infect such quiescent cells. The data supporting the present invention is limited to lentiviruses and accordingly the viruses suitable for this invention is limited to lentiviruses. Most preferably the lentivirus is based on the Human immunodeficiency virus, and perhaps preferably HIV 1. Other lentiviral vectors developed include those based on feline immunodeficiency virus (FIV) and equine infectious anaemia virus (EIAV).

The effectiveness of the dose of the lentiviral particle is relative to the integration event. It is hoped that a single delivery event will result in integration in sufficient numbers of epithelial cells to give the desired effect. The effect might therefore be measurable by the level of expression of the exogenous gene product, or by some effect that the gene product has. It is anticipated that not every cell of the target type needs to be infected, but simply a proportion.

The penetration enhancer might be selected from a range of materials that assist with the penetration of cells of the pulmonary system by the lentiviral particle.

The penetration enhancer might be one of a range of different types which act to enhance absorption into the layer of epithelial cells lining the lung. The enhancer can accomplish this by any of several possible mechanisms, including the following:

(1) Enhancement of the paracellular permeability by inducing structural changes in the tight junctions between the epithelial cells.

(2) Enhancement of the transcellular permeability by interacting with or extracting protein or lipid constituents of the membrane, and thereby perturbing the membrane's integrity.

(3) Interaction between enhancer and viral particle which increases the solubility in aqueous solution. This may occur by preventing formation of aggregates of the viral particles.

(4) Decreasing the viscosity of, or dissolving in part or full, the mucus barrier lining the alveoli and passages of the lung, thereby exposing the epithelial surface for direct absorption.

Enhancers may function by only a single mechanism set forth above, or by two or more. An enhancer which acts by several mechanisms is more likely to promote efficient absorption of viral vector than one which employs only one or two. For example, surfactants are a class of enhancers that are believed to act by all four mechanisms listed above. Surfactants are amphiphilic molecules having both a lipophilic and a hydrophilic moiety, with varying balance between these two characteristics. If the molecule is very lipophilic, the low solubility of the substance in water may limit its usefulness. If the hydrophilic part overwhelmingly dominates, however, the surface active properties of the molecule may be minimal. To be effective, therefore, the surfactant must strike an appropriate balance between sufficient solubility and sufficient surface activity.

One potential type of enhancer is the salt of a fatty acid. If the carbon chain length is shorter than about 8, the surface activity of the surfactant may be too low, and if the chain length is longer than about 16, decreased solubility of the fatty acid salt in water limits its usefulness. Different counterions may change the solubility of the saturated fatty acid salt in water, such that an enhancer having a carbon length other than 8-16 would prove even more advantageous than the enhancers specifically mentioned herein above. Salts of unsaturated fatty acids may also be useful in the present invention since they are more water soluble than salts of saturated fatty acids, and can therefore have a longer chain length than the latter and still maintain the solubility necessary for a successful enhancer of viral vector absorption.

The surface active agent might be a bile salt or a bile salt derivative (for example sodium salts of ursodeoxycholic acid, taurocholic acid, glycocholic acid, and taurodihydrofusidic acid). Examples of suitable bile salts include salts (e.g., sodium or potassium salts) of cholic acid, chenodeoxycholic acid, glycocholic acid, taurocholic acid, glycochenodeoxycholic acid, taurochenodeoxycholic acid, deoxycholic acid, glycodeoxycholic acid, taurodeoxycholic acid, lithocholic acid and ursodeoxycholic acid.

The surface active agent might be a phospholipid. It is found that a single-chain phospholipid (lysophospatidylcholine) is an effective enhancer. Examples of single-chain phospholipids include lysophosphatidylcholine, lysophosphatidylglycerol, palmitoylphosphatidylglycerol, palmitoylphosphatidylcholine, lysophosphatidylethanolamine, lysophosphatidylinositol, and lysophosphatidylserine. Certain double chain phospholipids may also be suitable. Examples of double-chain phospholipids include diacylphosphatidylcholine, diacylphosphatidylglycerol, diacylphosphatidylethanolamine, diacylphosphatidylinositol, diacylphosphatidylserine, and phosphatidylcholine, phosphatidylethanolamine, phosphatidylserine, phosphatidylinositol, phosphatidylglycerol, cholates, phosphatidic acid, and cardiolipin.

The surface active agent might be a glycoside for example the alkyl glucosides (e.g., decyl glucoside, dodecyl glucoside, and alkyl thioglucopyranoside) and alkyl maltosides (e.g., decyl maltoside and dodecyl maltoside).

The cyclodextrins and derivatives thereof effectively enhance the nasal absorption of insulin, and may have a similar function in the lung.

Other potentially useful surfactants are sodium salicylate, sodium 5-methoxysalicylate, and the naturally occurring surfactants such as salts (e.g., sodium and potassium salts) of glycyrrhizine acid, saponin glycosides, and acyl camitines such as decanoyl carnitine, lauryl carnitine, myristoyl carnitine, and palmitoyl carnitine.

For ionic enhancers (e.g., the anionic surfactants described above), the nature of the counterion may be important. In general, it is expected that monovalent metallic cations such as sodium, potassium, lithium, rubidium, and cesium will be useful as counterions for anionic enhancers. Ammonia and organic amines form another class of cations that is expected to be appropriate for use with anionic enhancers having a carboxylic acid moiety. Examples of such organic amines include ethanolamine, diethanolamine, triethanolamine, 2-amino-2-methylethylamine, betaines, ethylenediamine, N,N-dibensylethylenetetraamine, arginine, hexamethylenetetraamine, histidine, N-methylpiperidine, lysine, piperazine, spermidine, spermine, and tris(hydroxymethyl)aminomethane.

Surfactants may be alternatively be nonnatural compounds such as: polysorbates, which are fatty acid esters of polyethoxylated sorbitol (Tween); polyethylene glycol esters of fatty acids from sources such as castor oil (Emulfor); polyethoxylated fatty acid, e.g. stearic acid (Simulsol M-53); Nonidet; polyethoxylated isooctylphenol/formaldehyde polymer (Tyloxapol); poloxamers, e.g., poly(oxyethylene)poly(oxypropylene) block copolymers (Pluronic); polyoxyethylene fatty alcohol ethers (Brij); polyoxyethylene nonylphenyl ethers (Triton N); polyoxyethylene isooctylphenyl ethers (Triton X); and SDS. Mixtures of surfactant molecules, including mixtures of surfactants of different chemical types, may be acceptable. Surfactants should be suitable for pharmaceutical administration and compatible with the viral particle.

A transient tolerably damaging amount of penetration enhancer can be determined empirically. It is thought that in order for the penetration enhance to function properly it must allow passage of the lentivirus through the tight junction so that the lentivirus gains access to its receptors which are believed to be positioned away from the apical surface. Typically the surface epithelium present a negative potential difference when intact so that when the tight junctions are disrupted then the potential difference is reduced or erased. Thus the reduction in the potential difference has in the past been taken as a measure of integrity of the surface epithelial layer, and it is thus certain procedures are known to test for transepithelial potential difference (TPD). It is considered that should there be substantial exfoliation then the damage exceeds what the inventors consider as tolerable, and in which case it can take several day for the TPD to return to normal following substantial replacement of the surface epithelium. The return of TPD to normal where substantially only the tight junctions are disrupted might take less than about 2 days and preferably only several hours perhaps 6. Ideally it may be desired to have the cells return to normal shortly after transduction with lentivirus.

For LPC (lysophosphatidyl choline) it is thought that, for example, a concentration of 0.1% is still effective however a lesser amount may still give an adequate effect, thus for example the amount LPC used might be about 0.01%. It is thought that larger amounts might also still be effective and sufficiently non-damaging, thus whilst an amount of 2 to 3% may have a damaging effect this can still provide sufficiently integration ready cells. It is thought however that delivery of 5% LPC is unacceptably damaging. It might be that there may be a requirement that delivery of the viral particle may need to be delayed further with higher concentration to allow cells so treated to recuperate. Thus with lesser amounts of penetration enhancer, for example 0.1% LCP it might be desired to deliver virus particles in less than 1 hour and perhaps most preferably at the same time, with larger amounts of penetration enhancer the delay might preferably be more like 12 hours. It is presently thought that a delay between delivery of the penetration enhancer and the viral particle is desirable although simultaneous delivery may still be effective. Additionally the delay may also relate to the condition of the mammal to which it is delivered. Thus where there is rapid build up of mucus in the lung it might be desired to allow adequate time for clearance but not allow for a longer delay to allow mucus build up again.

Although the in vivo gene transfer studies described for this invention were performed in the nasal airway, it is clear that the LV-based gene delivery protocol must be further developed for gene delivery to the conducting airways. The two obvious methods for LPC (or vector) administration into the conducting airways, which could be employed in a clinical setting, are bolus instillation via a bronchoscope or nebulisation. Instillation of the LPC (or the viral vector) via a bronchoscope allows the delivery of the solution to a specified area in the lung. However, this method of instillation can expose the alveolar space to respiratory debris, —causing alveolar contamination—, consequently resulting in the generation of an inflammatory response [Joseph, 2001 #416]. In contrast, nebulisation is a relatively simple technique and more convenient in a clinical setting than the liquid instillation approach. However, the administration of the solution cannot be localised to a specified area. As a consequence, the oesophagus, the mouth and the nasal airways are also exposed to LPC. In addition, the efficiency of this technique depends heavily on the breathing pattern of the individual patient. Another method of administration, which has been recently described, relies on delivering small volumes of liquid as large droplets, allowing for the instillation of solutions to a specific site in the airways [Cipolla, 2000 #422]. This method appears to allow precise deposition of the solution to a specific area in the airways without causing alveolar contamination.

The viral particle may also incorporate targeting molecules such as antibodies, lectins and other molecules that specifically bind receptors of the cells to which the gene therapy is applicable, such targeting molecules might be expressed on the surface of the viral particle.

Preferably the method further includes the step of monitoring the expression of the gene in the respiratory system. Such measurement might be direct or indirect. Thus for example one might measure the production of a particular product which might thus be achieved by immunological testing for the presence of the product, either in situ or after sampling. Alternatively and preferably where the product is to ameliorate a condition then the measurement will be an indicator of that condition.

Thus in the case of cystic fibrosis pulmonary ΔPD is an indicator of the expression of the CFTR gene. Additionally indicators are physical symptoms of the pulmonary manifestation of CF, for example congestion. Similar clinical indicators or physically measurable parameters can also be used where other conditions might be employed.

The invention might also in this broad aspect be said to reside in a composition including a penetration enhancer and a lentivirus particle carrying an exogenous gene in a suitable carrier and/or excipient suitable for respiratory inhalation or bronchial spray. In a preferred form the exogenous gene encodes CFTR, and in a preferred form the penetration enhancer is LPC. Additionally more preferably the lentivirus is a modified HIV-1 particle.

One finding of the present invention of particular interest is in relation to the cells in which expression marker used in this invention was found. The enzyme lacZ was found expressed into a large range of cells, and of greater interest was that expression in certain of the cells was not found until late time samplings. Thus lacZ was not found in the secretory cells known as goblet cells until 92 days. This taken together with the persistence suggest that transduction was effected in cells that were non-terminally differentiated cells. These cells are a progenitor cell of some type and it is postulated by the inventors that the use of LPC together with lentivirus has accessed for the first time progenitor cells and perhaps stem cells. This then would account for the persistence that has been found whereby the exogenous gene has been found to be expressed for periods longer than 46 days, 92 day and 13 months. The physiological effect for amelioration of the CFTR gene defect was also found to last longer than 46 day and 92 day and in certain test animals for greater than 110 days.

It is anticipated that the present invention will be applicable across a range of mammals. These include but are not limited to humans, agricultural, sporting and domestic mammals, which might include animals such as racehorses cats and dogs.

### EXAMPLE 1

Persistence of Gene Therapy Product In Vivo

CF is the most common life threatening, autosomal recessive disease in Caucasian populations, especially those of Northern European origin (Hodson and Geddes, 1995). Although many organs are affected by the ion imbalances induced by malfunction of the cystic fibrosis transmembrane conductance regulator (CFTR), it is the chronic and progressive infective lung disease that produces the high levels of mortality and morbidity experienced by CF children and adults. Recent studies indicate that the primary effect in the CF airway of the derangement of epithelial cell ion balance is a reduced airway surface liquid (ASL) volume (Tarran et al., 2001) that ultimately results in defective mucociliary clearance, allowing inhaled bacteria to deposit, proliferate, and initiate a chronic infective/inflammatory lung disease (Knowles and Boucher, 2002).

Gene therapy for CF lung disease is based on the premise that if adequate CFTR function can be restored in the defective CF airway epithelial cells then airway epithelial biology and overall lung function would be normalised. Airway infection should then be prevented and morbidity and mortality associated with CF lung disease averted. If airway progenitor cells could also be permanently transduced then therapeutic CFTR gene expression would be sustained. Of the viral gene transfer vectors currently being developed, LV vectors have particular advantages in that they can transduce both quiescent and dividing cells, provide stable and sustained gene expression, and do not appear to induce a significant host immune response (Amado and Chen, 1999).

While the conceptual basis of gene therapy for CF lung disease using gene transfer is both elegant and simple, and is easily demonstrated in vitro (Johnson et al., 1992), practical barriers to its application in vivo have become apparent (Koehler et al., 2001; Davies et al., 2001). The highly effective airway defences that have evolved to protect the mammalian airway epithelium against allergens, irritants, dust, viruses and microbial pathogens (Bevins et al., 1999) also apply to gene transfer vectors. Airway mucus continually captures inhaled or introduced particles for removal by mucociliary clearance activity. At the apical cell surface the glycocalyx (Pickles et al., 2000) can bind most vector types, further hindering vector particle entry. Finally, the epithelial tight-junctions (TJ) present another physical barrier to the delivery of gene transfer vectors to their receptors, located predominantly on the basolateral cell surfaces below the TJ (Bergelson et al., 1997). Modulating the effectiveness of these barriers, for example by opening airway epithelial TJ to facilitate access of gene transfer vector particles to receptors located on the basolateral cell surface, is an approach to improving in vivo gene transfer that has only recently received attention (Parsons et al., 1998; Johnson et al., 2000, Wang et al., 2000; Coyne et al., 2000; Chu et al., 2001). Our focus has been to use surface active agents such as the synthetic detergent polidocanol (Parsons et al., 1998) and the biological detergent lysophosphatidylcholine (LPC) (Parsons et al., 1999), to pre-condition the airway surface to make it temporarily permissive for viral gene transfer in vivo.

In this example we show that LPC, used as pre-treatment reagent to condition the airway epithelium surface, permits sustained gene transfer of a VSV-G pseudotyped HIV-1-based LV vector into mouse nasal airway epithelial cells in vivo. Furthermore, a single LV vector dose was sufficient to produce expression of a marker gene and a therapeutic gene that outlasted the generally accepted turnover time (˜3 months) of the airway epithelium (Borthwick et al., 2001), suggesting that transduction of airway progenitor cells had occurred.

The mouse model has been used in this example, because the mouse nasal airway is considered to be the most approapriate current model for developing and testing gene vectors for CF.

Materials and Methods

DNA Construction and Virus Production

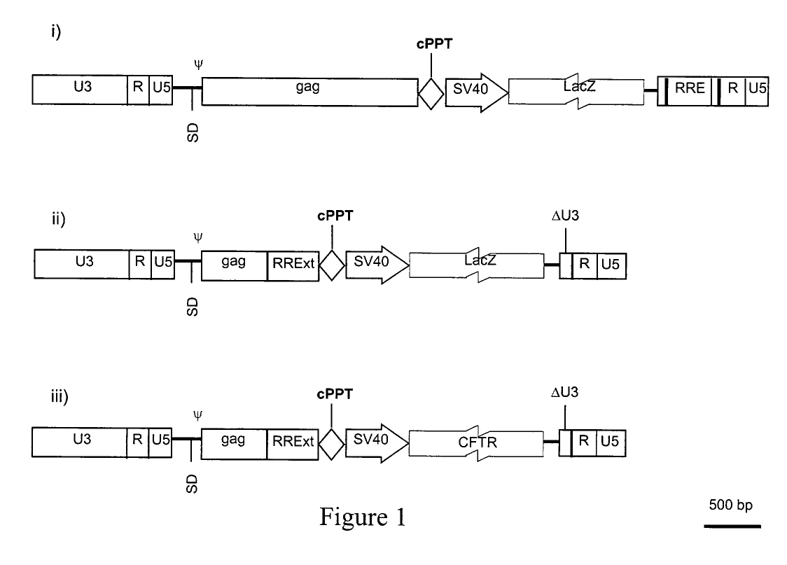

The LV vector system used in this study (Fuller and Anson, 2001; Anson and Fuller 2001)) has been derived from HIV-1 and has been disassembled to prevent viral replication. The LV vector was produced by transient transfection of 293T cells with five different plasmids including 14 μg of the LV vector plasmid, 3 μg of pcDNA3gagpolml, 14 μg of pHCMV-G (Yee et al., 1994), 4 μg of pCMV-rev (Lewis et al., 1990) and 0.2 μg of pcDNA3TAT101 ml using the calcium phosphate co-precipitation transfection protocol (Fuller and Anson, 2001). Three different LV vector constructs were used, (i) pB1HIVext5cpptSV40LacZppt⁺RRELTR (LVLacZa), (ii) pBCKSHIVext4crrextcpptSV40LacZppt⁺ΔLTR (LVLacZb), and (iii) pBCKSHIVext4 m2crrextcpptSV40CFTRppt⁺ΔLTR (LVCFTR) (FIG. 1). The LVLacZa and LVLacZb vectors contain essentially the same HIV-1 sequence elements but differ slightly in the arrangement of these sequences. The most notable difference is the shorter length of gag gene sequence in the latter vector. The LVCFTR vector construct is essentially the same as the LVLacZb vector but with the CFTR cDNA sequence replacing the LacZ marker gene sequence. In all three vectors the transgene is under the transcriptional control of the simian virus 40 (SV40) early promoter. The LV vector supernatant collected was initially concentrated ˜10-fold by ultrafiltration in a 50 ml stirred cell apparatus using a DIAFLO® Ultrafiltration membrane (500K weight cut off, ZM500, Amicon, Inc., USA) at 4° C. and further concentrated (˜100-fold) by ultracentrifugation at 30,000×rpm for 1 h and 35 min at 4° C. in a Beckmann SW-60 rotor. The resulting LV vector pellet was typically resuspended in 200-300 μl of PBS (1/1000 volume of the starting volume of the LV vector supernatant) and stored at −70° C.

Estimation of the Titre of the LV Vector

LVLacZ vector: NIH3T3 cells grown on 6-well tissue culture clusters were transduced with either the LVLacZa vector, or the LVLacZb vector in the presence of polybrene (4 μg/ml). Seven days later the transduced cells were stained with 1 ml of X-gal solution (Parsons et al., 1998) for 16 h at 37° C. to detect LacZ gene expression. The X-gal solution was then aspirated and washed once with PBS. For each well the number of LacZ positive cells in three 0.25 cm² squares was counted. The titre (transducing units: TU) of the LVLacZa and the LVLacZb vectors was calculated using the following formulae:

Number of LacZ positive cells×dilution factor×total surface area (9.4 cm²)/selected surface area (0.75 cm²)=NIH3T3-TU/ml

LVCFTR vector: DNA was prepared from non-transduced A549 cells (control) and A549 cells TM transduced with either the LVCFTR vector, or a vector of known titre, using the DNeasy™ tissue kit (QIAGEN, Germany) according to the manufacturer's instructions. The primers Ext 4F (5′ GGGTGCGAGAGCGTCAGTATTAG 3′) and Ext 4R (5′ CTTCTCTAAAGCTTCCTTGGTGTC 3′) (GIBCO, Australia) were designed to amplify a 306 base portion (HIV-1 YU-2, GenBank accession number M93258, bases 803-1109) of the gag gene sequence in the vector. Dilutions of sample and positive control (known amounts of plasmid DNA and/or DNA prepared from cells transduced with a vector of known titre) were amplified in the presence of 1×Taq buffer (Qiagen, Germany), 2.5 mM MgCl₂, 2×Q buffer (Qiagen, Germany), 200 μM dNTPs (Roche, Australia), 1 μg of Ext4 F and Ext4 R primer and 5U of Taq (Qiagen, Germany). The reaction was heated initially to 94° C. for 3 min, followed by 35 cycles of, denaturation at 94° C. for 30 sec, annealing at 60° C. for 60 sec and extension at 72° C. for 30 sec. The last cycle was a further extension at 72° C. for 3 min. PCR products were then analysed by agarose gel electrophoresis and titre calculated by comparing the amount of product from the dilutions of the experimental sample with that from the dilutions series of the positive control. It should be noted that the titre of the LV assayed on A549 cells is 10-20 times higher than that assayed on NIH3T3.

Assay for Detection of Helper Virus

In order to monitor for replication competent retrovirus the p24 levels present in the medium of transduced A549 cells were monitored. Briefly, A549 cells were transduced with a sample of the concentrated LV vector (LVLacZa, LVLacZb, or LVCFTR) and maintained in culture for 4 weeks. Twice a week a 1 ml sample from a confluent culture was collected and stored at minus 70° C. until all samples were collected. The medium of non-transduced A549 cells was also collected and used as a negative control. The samples were assayed for p24 using the HIV-1 p24 ELISA kit (NEN Life Sciences, USA) according to the manufacturer's instructions. In all cases p24 declined to undetectable levels (<10 pg/ml) within 7 days of transduction.

Nasal Dosing

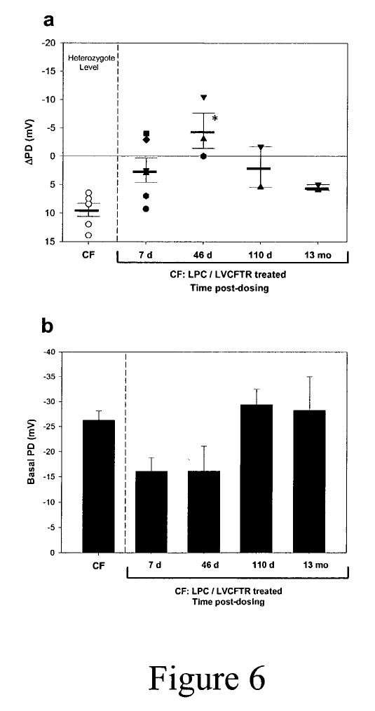

C57B1/6 and cftr^{tmlUnc} mice (Snouwaert et al., 1992) were used under the approval of both the Women's and Children's Hospital and the University of Adelaide Animal Ethics Committees. C57B1/6 mice (6-7 weeks of age), or cftr^{tmlUnc} mice (8-20 weeks of age) were anaesthetised intramuscularly with 1 μl/g and 0.7 μl/g body weight respectively of a 3:2 mixture of xylazil (20 mg/ml):ketamine (100 mg/ml) respectively. Body temperature was maintained during anaesthesia with a heat pad, or heat lamp and during the recovery period the mice were placed in a 35° C. air chamber. For dosing, mice were suspended from their dorsal incisors (hindquarters supported) and pre-treatment solutions (4 μl of LPC (Sigma L-4129), or polidocanol (Sigma P-9641), prepared as w/v solutions in PBS) delivered as a bolus into the right nostril using a gel-loading tip (Finnpippette). Typically, thirty minutes after the initial anaesthetic dose, mice were re-anaesthetized with half the starting anaesthetic dose. One hour after the detergent pre-treatment, the LV vector (or the appropriate control solution) was instilled. Two 10 μl aliquots were instilled in the right nostril over 2-3 minutes. The mice were monitored for respiratory distress and any loss of treatment solution was noted. Mice were weighed daily for 10 days, and observed for signs of distress over the duration of the experiment. Deaths of LV-treated CF mice in this series (FIG. 6a) were due to complications during post-anaesthesia recovery.

Assessment of Gene Transfer

LacZ gene expression: The heads were processed to reveal LacZ gene expression using X-gal processing as previously described (Parsons et al., 1998). The types of LacZ positive cells in respiratory and transitional epithelium were determined in H/E stained cross-sections, while the number of LacZ transduced cells was counted in 3 standard cross-sections (Parsons et al., 1998) stained with Safranin (Saf-O).

CFTR gene expression: Mice were anaesthetised, suspended from their dorsal incisors (hindquarters supported) and a subcutaneous needle-agar bridge (as reference electrode) was placed in the abdomen. A heat-drawn PE10 polyethylene cannula (marked with a fine tip permanent marker at 2.5 mm and 5.0 mm to allow accurate placement of the cannula tip) was inserted to the designated depth in the treated nostril and connected to a perfusion-recording apparatus (modified dual syringe pump (IVAC 770), a WPI Isomillivoltmeter and a chart recorder). The syringe pump was loaded with two 1 ml Hamilton syringes (Hamilton Instruments, NV, USA) containing either basal or low-chloride solution and connected to the tubing system. The cannula was inserted into the treated right nostril of the mouse at ˜3 mm (this depth was 2 mm shallower than that used in previous studies (5 mm) (Parsons et al., 1998) in an effort to improve the recording of electrical potential from only respiratory epithelium in the nose (Parsons et al., 2000a). Infusion of the basal solution (˜2.3 μl/min) was initiated and readings were taken until a stable TPD value was recorded (a plateau of at least 1 minute was required). The infusion solution was then switched to low-chloride solution (NaCl replaced with Na gluconate) and a new TPD value recorded. Two untreated cftr^{tmlUnc} mice were also used for blinding purposes and as negative controls at each TPD assessment point (7, 46, 110 days and 13 months, data not shown). The treatment category of the cftr^{tmlUnc} mice was blinded by tail colour re-coding prior to TPD recordings. TPD values were measured (blinded) from chart paper recordings, and the ΔPD value was calculated by subtracting the TPD value recorded under basal conditions from the TPD value measured under low-chloride conditions.

Statistical Analysis

Statistical analysis of data was performed using SigmaStat 2.03 (SPSS, Chicago, Ill.). Statistical significance was set at P=0.05 and a statistical power greater than 0.80 was required (if power did not reach 0.80 it is noted). Data are presented as a mean±standard error (SEM). Student's t-test was used for two-group comparisons, and multiple treatment groups were analysed by one-way ANOVA analysis using post-test multiple comparisons to identify specific group differences. Where data did not satisfy normality assumptions standard transformations or appropriate non-parametric methods were utilised. Changes in the proportions of transduced cell types were analysed by logistic regression analysis using GenStat, Release 4.2, 5^{th} Edition (VSN International Oxford, UK).

Results

LV-Mediated LacZ Gene Transfer into the Nasal Airway Epithelium

To determine the most effective detergent pre-treatment regimen we first compared the effect of conditioning treatment with two doses of either polidocanol, or LPC, on the level of in vivo LacZ marker gene transduction. Groups of mice (n=3) were exposed to either polidocanol (1% or 0.1%), or LPC (1% or 0.1%) one hour prior to instillation of 20 μl of either the LVLacZa vector (FIG. 1) containing 6×10⁴ NIH3T3-TU, or the carrier solution (PBS). To assess the effect of polybrene on LV-mediated gene transfer a further group of mice were pre-treated with 1% LPC and exposed to the same dose of LVLacZa vector containing 4 μg/ml polybrene. Seven days later, LacZ gene expression in the nasal epithelium was revealed using the X-gal processing method.

Qualitative stereo-microscope en face examination of the grossly-sectioned blocks of the head (prior to paraffin embedding and histological processing) showed that 1% LPC facilitated significant gene transfer compared to PBS pre-treatment. The distribution of the LacZ-positive cells, identified as scattered punctate blue-stained cells, remained ipsilateral, whereas a diffuse light green artefactual staining was distributed bilaterally. No LacZ positive cells were seen in the control (PBS pre-treated) mice. In addition, no LacZ positive cells were observed in olfactory regions.

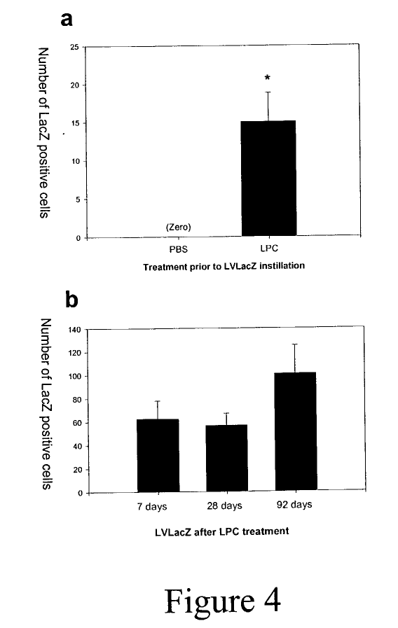

When the number of LacZ positive cells was quantified in nasal cross-sections several conclusions could be made regarding our LV-mediated gene transfer protocols Firstly, 1% LPC pre-treatment produced a 4-fold increase in the level of LV-mediated transduction compared to pre-treatment with 1% polidocanol, and overall the 1% LPC pre-treatment provided significantly greater gene transfer than other pre-treatments (ANOVA, P<0.05, Table 1) including PBS (control) pre-treatment (FIG. 4a). Secondly, inclusion of polybrene in the LVLacZa vector preparation resulted in a 4-fold decrease in the number of LacZ positive cells (5.0±1.0 versus 19.7±1.7, P=0.002). These results indicated that LPC was a more effective pre-treatment reagent than polidocanol, and that the addition of polybrene reduced the level of LV-mediated gene transduction in this in vivo setting. Therefore, airway conditioning with 1% LPC, and polybrene-free LV vector preparations, were used in all subsequent in vivo gene transfer studies.

TABLE 1
Total number of the LacZ positive cells.

| Pre-treatment | | Treatment | LacZ positive cells ± SEM |
|---|---|---|---|
| | PBS | LVLacZa | 0.0 ± 0.0 |
| 1% | PDOC | PBS | 0.0 ± 0.0 |
| 1% | LPC | PBS | 0.0 ± 0.0 |
| 0.1% | PDOC | LVLacZa | 2.7 ± 0.3 |
| 0.1% | LPC | LVLacZa | 4.3 ± 0.7 |
| 1% | PDOC | LVLacZa | 5.3 ± 1.3 |
| 1% | LPC | LVLacZa | *19.7 ± 1.7 |

C57B1/6 mice (n=3 per group) were pre-treated and dosed with 20 μl of LVLacZa containing 6×10⁴ NIH3T3-TU as described in Materials and Methods. Results are presented as the number of LacZ positive cells counted in 3 standard cross-sections. The combination of 1% LPC pre-treatment and LVLacZa produced the greatest number of LacZ positive cells in nasal airway in vivo (P<0.05, ANOVA). In addition, to the above results no LacZ positive cells were found in two control groups of mice (n=3 each) tested by pre-treatment with 4 μl LPC pre-treatment followed by (a) 20 μl of LVEYFP (enhanced yellow fluorescent protein, an irrelevant reporter gene in this context); and (b) a LacZ pseudotransduction control (pcDNALacZ ‘virus’) in which a plasmid expressing LacZ was substituted for the LVLacZa.

Persistence of LacZ Gene Expression into the Nasal Airway Epithelium

The level of persistence of gene expression will be a critical determinant of the utility of a vector in producing effective therapeutic CFTR gene transfer in CF airway epithelium. Therefore, we assessed the persistence of gene expression resulting from LV-mediated transduction in nasal airway epithelium. The right nostril of 9 mice was exposed to 1% LPC 1 hour prior to the instillation of 20 μl of LVLacZb (FIG. 1), containing 1.4×10⁵ NIH3T3-TU. Three treated mice were subsequently sacrificed at 7, 28 and 92 days and the heads processed for LacZ marker gene expression.

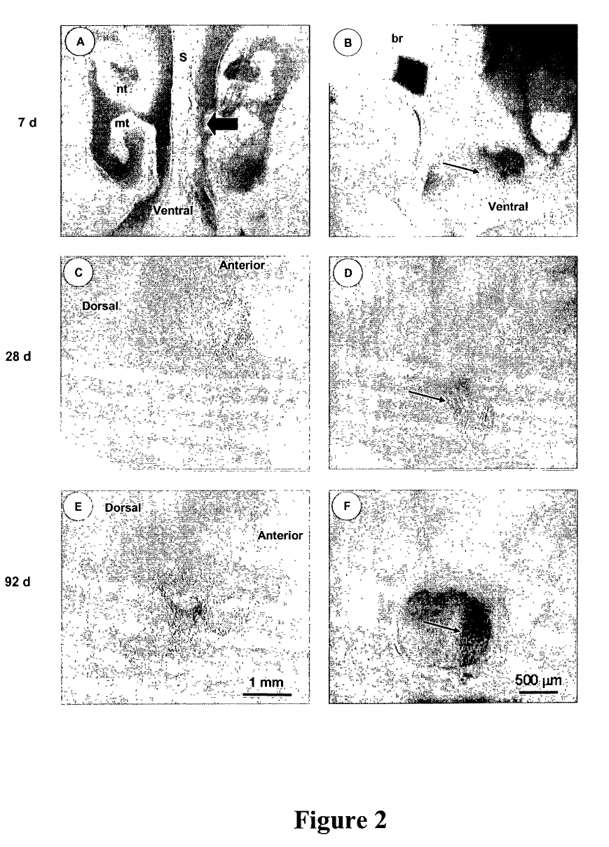

Qualitative en face examination of the blocks of tissue of the nasal airway epithelium at each post-treatment time point revealed high numbers of LacZ positive cells. The distribution of these LacZ positive cells at each time-point remained ipsilateral along the treated nostril (FIGS. 2A, C, E) and there were also high numbers of LacZ positive cells located as far posterior as the nasopharyngeal meatus (FIGS. 2B, D, F). Interestingly, although the two nasal airways (one dosed, one un-dosed) have coalesced into the single nasopharyngeal airway at this posterior level of the nose, LacZ positive cells remained localised to the dosed side of the head. Again, no LacZ positive cells were seen in the olfactory regions.

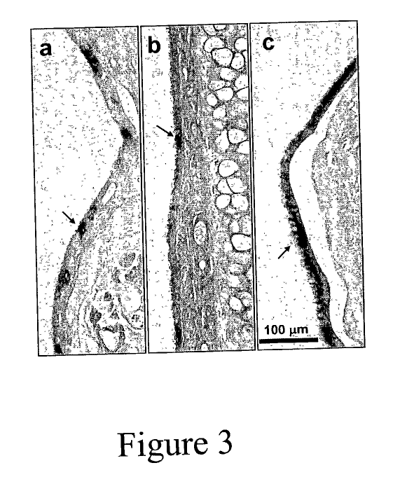

When the number of LacZ positive cells was quantified in Saf-O stained cross-sections (FIGS. 3a, b, c) the number of LacZ positive cells observed on days 7, 28 and 92 post-treatment was maintained at similar levels (FIG. 4b) confirming the persistence of expression produced by our LV-mediated gene transfer protocol.

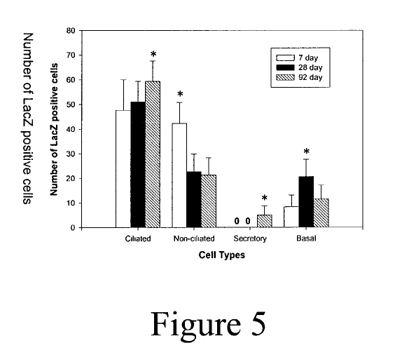

It is important that the appropriate airway epithelial cell(s) are transduced when developing gene transfer protocols for CF gene therapy (Parsons et al., 2000a). LacZ positive cells were observed in all regions of the nasal airway with the exception of the olfactory and squamous regions. Quantitative determination of LacZ positive cells was restricted to areas of respiratory and transitional epithelium, which both contain ciliated cells. The types of transduced cells were determined in H/E-stained sections. We found that transduced cells were predominantly ciliated and non-ciliated; smaller numbers of transduced secretory (predominantly goblet) and basal cells were also seen. The numbers of each cell type showed significant changes over the duration of the experiment (FIG. 5). Of note was that LacZ positive secretory cells were only observed at 92 days post-treatment.

Correction of the CFTR Defect in the cftr^{tmlUnc} Mice

Six cftr^{tmlUnc} mice were exposed to 1% LPC one hour prior to the instillation of the LVCFTR vector containing 1×10⁷ A549-TU. As a control for the effect of LPC alone, three cftr^{tmlUnc} mice were exposed to 1% LPC one hour prior to the instillation of carrier solution (PBS).

Decreasing (more negative) ΔPD values reflect increasing functional correction of defective CFTR-mediated chloride secretion (Parsons et al., 1998). Untreated cftr^{tmlUnc} mice exhibited a ΔPD value of +9.5±1.2 mV compared to heterozygote CF colony mice (−16.5±2.0 mV). Treatment of homozygote cftr^{tmlUnc} mice with LPC prior to PBS (control) instillation did not alter the ΔPD value when examined 7 days later (+8±3.2 mV).

Seven days after exposure to the LVCFTR vector functional expression of CFTR in cftr^{tmlUnc} mice resulted in more negative (but not significantly different) mean ΔPD value (+2.5±2.2 mV) compared to those of untreated cftr^{tmlUnc} mice (FIG. 6a). Forty-six days post-treatment functional recovery of CFTR activity had reached 54% of the mean heterozygote ΔPD value, a statistically significant improvement (ΔPD=−4.5±3.1 mV, p<0.05 ANOVA, Dunnets multiple comparison) from the (control) untreated CF mouse ΔPD value. At one hundred and ten days after LVCFTR vector instillation partial functional CFTR correction had persisted in one of the two surviving LV-treated cftr^{tmlUnc} mice (ΔPD=−1.7 mV), whilst in the second mouse the ΔPD had waned (ΔPD=+5.5 mV). By thirteen months the ΔPD values in both these mice had declined further (ΔPD=+5.0 mV and ΔPD=+6.0 mV; 17% and 13% of mean heterozygote ΔPD values, respectively).

The effect of the LVCFTR vector instillation protocol on the basal TPD values is shown in FIG. 6b. The apparent reductions in TPD present at 7 and 46 days did not reach statistical significance.

Discussion

For gene therapy to evolve as a suitable treatment for CF lung disease ways of improving the historically low efficiency of gene transfer to airway epithelium must be found. Modulating the airway epithelium to enhance gene transfer, for example by altering TJ permeability to improve access of vectors to their receptors on basolateral surfaces, is an approach that has only recently received attention. Improving basolateral access should also allow enhanced gene transfer to sub-apical progenitor cells (Engelhardt, 2001). Successful transduction of progenitor cells would be expected to result in the generation of a stable population of gene corrected airway epithelial cells over long time frames. Although airway progenitor cells have recently been identified in the rodent trachea (Borthwick et al., 2001), the identity of progenitor cells in human ciliated airway epithelium remains controversial.

Increased efficiency of gene transfer following modulation of airway TJ has already been demonstrated. For example, treatment with EGTA (a Ca²⁺ chelator) in a hypotonic buffer opens epithelial TJ and improves the efficiency of in vivo gene transfer by adenovirus (AdV) (Wang et al., 2000; Chu et al., 2001), adeno-associated virus (Duan et al., 1998), and retrovirus (Wang et al., 2000) vectors. In contrast to the relatively slow action of EGTA (1 h to achieve opening of TJ), apical application of sodium caprate on well-differentiated airway epithelial cells in vitro resulted in the rapid opening of the airway epithelial TJ, also improving AdV-mediated gene transfer (Coyne et al., 2000). In addition, exposure of the mouse nasal epithelium to the toxic gas SO₂ prior to the instillation of a VSV-G pseudotyped LV vector expressing the LacZ marker gene improved the efficiency of LV-mediated gene transfer in vivo (Johnson et al., 2000).

Our modifications to the detergent-based procedures originally developed for the AdV vector (Parsons et al., 1998) have shown that pre-treatment of mouse nasal airway epithelium with LPC considerably enhances the capability of our LV vector to transduce intact airway epithelium. A key finding of this study was that exposure to a single dose of a LV vector carrying the CFTR gene could produce significant electrophysiological recovery of CFTR function in cftr^{tmlUnc} mouse nasal airway for at least 110 days. The gene transfer was not observed unless LPC conditioning was employed and the inventors believe the use of LPC (or other surface-active agents) is critical.

LPC is the detergent component of pulmonary surfactant (comprising 2-5% of total phospholipids (Niewochner et al., 1989; Weltzien et al., 1979). Apart from the TJ modulation effects of LPC (Parsons et al., 1999) there are several potential mechanisms by which LPC could modulate the airway epithelium. The mucolytic properties of LPC could solubilise airway mucus by reducing its viscosity and elasticity (Martin et al., 1978); LPC should also reduce MCC activity since it is able to reduce cilial beat (Merkus et al., 1993); indeed, we have observed dose-dependant reversibility of reduction in cilial beat in freshly-excised mouse nasal airway epithelium (M. Limberis, unpublished data). In pilot studies LPC has improved surrogate vector particle deposition onto airway epithelium in vivo (Parsons et al., 2000b). While there are likely to be other biological effects of LPC (Prokazova et al., 1998) that may be relevant to gene transfer, the direct airway surface effects described here would each be expected to contribute to enhanced gene transfer in vivo, by improving retention of gene transfer vector particles after deposition, and subsequent access to the basolateral cell surface.

Ideally, a pre-treatment/conditioning agent should produce a transient and tolerable perturbation of the barrier function(s) of ciliated airway epithelium. Histological injury that has been observed immediately after dosing with 1% LPC alone, i.e. limited areas of deciliation or exfoliation found anteriorly close the dosed site (D W Parsons, unpublished), and this same LPC exposure prior to LV vector instillation was associated with effective LacZ, or CFTR gene transduction (FIG. 4a, b; FIG. 6a). However, we also found persisting LacZ gene expression in nasal epithelium as far posterior as the nasopharyngeal meatus (FIGS. 2B, D, F) a site where LPC-induced cell injury was not observed. It thus appears the more dilute concentrations of LPC reaching this region also permit LV-mediated gene transfer. This finding is consistent with the data showing that milder (non-injurious) LPC-based airway modulation is also effective in enhancing the effectiveness of other gene transfer vectors (Parsons et al., 1999). Endogenous LPC is rapidly converted in cell systems (Besterman and Domanico, 1992) and lung alveoli (Seidner et al., 1988) to the ubiquitous and non-toxic dipalmitoylphosphatidylcholine (DPPC), a primary component of biological membranes. We speculate that there is, therefore, some capacity for exogenous LPC to be similarly converted in vivo, providing a measure of active removal of the penetration enhancer that is not a feature of other airway barrier modulation reagents reported to date.

Interestingly, we found that the widely-used enhancer for retroviral vectors—polybrene (Coelen et al., 1983)—appears to inhibit LV-mediated gene transfer when used in vivo. This finding highlights the need to question assumptions generated from in vitro experiments when progressing to in vivo trials.

Since permanent expression of functional CFTR in CF airways is the primary goal in efforts to develop a cure for CF lung disease, the greater than three month persistence of LV-mediated LacZ gene expression we found after a single vector administration was encouraging. The number of LacZ positive cells showed an increase (non-significant) at 92 days (FIG. 4b). As the cell turnover time of rodent airway epithelium is thought to be in the order of 3 months (Borthwick et al., 2001), the total number of LacZ positive cells observed should have dropped substantially by 92 days unless airway progenitor cells had been transduced. In support of the belief that progenitor cells were transduced, we noted that although LacZ positive secretory cells were not seen at 7 or 28 days, they were present at 92 days (FIG. 5). This suggests that outgrowth and differentiation of (transduced) progenitor cells into secretory cells may have occurred between 28 and 92 days. Given the current rudimentary understanding of stem cell identity and physiology in airway epithelium the reason for the changes in the numbers of LacZ positive ciliated, non-ciliated and basal cells across the three assessment time-points cannot be addressed here. Presumably, the changes observed represent a dynamic balance between turnover of mature cells, and their replacement by outgrowth and differentiation of various progenitor populations, each of which will display a different initial transduction efficiency.

We did not specifically examine the effect of our LVCFTR gene transfer protocols on the sodium hyperabsorption that is characteristic of CF airway dysfunction (via comparison of airway PD values in amiloride supplemented/free perfusion solutions). Nevertheless, the (non-significant) reductions in mean basal PD value (an index of sodium channel activity) apparent 7 and 46 days after LVCFTR vector instillation suggested the LVCFTR dosing protocol might be able to alter the basal TPD. However, the values present at the later time points are clearly no different to the mean basal TPD values present in untreated CF mice. Additional studies that include appropriate TPD comparisons using amiloride-supplemented solutions are indicated, to both improve statistical power and to directly examine how CF airway sodium hyperabsorption is altered by LVCFTR vector exposure.

Correction of the electrophysiological defect in the ΔPD value of CF mice by LVCFTR gene transfer appears to have begun to decline by 110 days in this study. Several factors may contribute to this apparent difference in persistence in expression of the LacZ and CFTR genes. First, the measurement of nasal TPD in mice has inherent technical limitations (Parsons et al., 2000a) and we believe that such limitations may contribute to the variability observed in TPD values at each assessment time point. In particular, at each TPD assessment the cannula tip may not sample from precisely the same area of airway epithelium. The complexity of the mouse anterior nasal anatomy (Parsons et al., 2000a), the relative positional changes in nasal anatomy that accompany growth and the variability inherent in nasal cannula insertion procedures performed many weeks or months apart mean that larger numbers of mice will be needed to overcome this source of variability in any studies employing re-assessment of functional CFTR gene expression in nasal airway over time. Second, differences in the completeness of sampling of gene expression may be important. LacZ gene expression provides a visible and unambiguous assessment applicable to both the entire nasal airway (FIG. 2) and to standard samples of airway (FIG. 3), whilst measurement of CFTR gene expression samples a restricted area of airway epithelium under the TPD cannula tip.

Nevertheless, the partial correction of the electrophysiological defect resulting from LV mediated delivery of the CFTR gene in CF mice diminished between 46 and 110 days in this study, and had almost entirely disappeared by 13 months. The reasons for the discrepancy in gene expression persistence produced by the LVLacZ and LVCFTR vectors is not known; however, we note that neither the cell types requiring CFTR correction, nor the cells that produce the electrophysiologically-measured changes in epithelial TPD, are described for intact airway. The link between the ΔPD value, the level of CFTR expression per cell, and the percentage of cells expressing vector-delivered CFTR is also unknown in vivo. Understanding this relationship should provide key information to help direct the development of more efficient airway gene vectors; clearly, longer-term detailed studies using both the LacZ marker gene and the CFTR gene will be required to resolve these issues.

Since CF lung disease takes many years to establish, and because it is resistant to current therapies and often includes pathologies not directly related to CFTR dysfunction (e.g. airway wall damage subsequent to chronic bacterial infection), recovery of CFTR function alone is unlikely to produce immediate restoration of lung function in already-diseased lungs. Gene therapy for CF lung disease will therefore be targeted to the early childhood period prior to the acquisition of lung infection. Before this approach could be considered, parents and CF patients, researchers, and clinicians must be satisfied with the safety profile of both the gene transfer vector(s) and of any airway-conditioning reagent(s) used. However, our demonstration of persisting in vivo CFTR gene transfer after simple dosing procedures, and the recent developments in targeting and potential dosing simplicity provided by novel LV envelope pseudotypes (Kobinger et al., 2001) offer hope that the promise of LV gene therapy (Friedmann, 2000) can indeed be translated into a safe and effective treatment of CF lung disease. The simplicity of our transduction protocol, which utilises brief single exposures to LPC and LV vector, should facilitate further development towards clinical applications.

### EXAMPLE 2

Use of Different Detergents.

Other detergents have also been used to effect transfection for example e.g. polidocanol (PDOC, also known as polyoxyethylene 9-lauryl ether, and nonaetheylene glycol monodecyl ether), and the detergent SDS. These have been shown to permit transfection of cells leading to gene expression of the indicator lacZ. The methods use are in line with the methods used above.

The detergents tested were Sodium n-dodecyl sulfate (SDS), (an anionic detergent) at 0.25%, Zwittergent 3-14: N-Tetradecylsulfobetaine (ZWIT) (a zwitterionic detergent) at 0.14%, Cetyltrimethylammonium bromide (CTAB) (an ionic detergent) at 0.15%, Deoxycholic Acid (DEOXY) (an ionic detergent) at 0.17%, with phosphate buffered saline as a control.

Detergents were made up fresh to nominated concentrations. A single 4 μl dose was used per mouse (all were approximately 20 gm body weight). An Adenoviral vector (AdlLacZ) dose of 1×10⁹ pfu in 20 μl. Mice were anaesthetised with Xylamine/Ketamine i.m.; then suspended vertically (hanging on dorsal incisor teeth). The detergent dose was instilled into the nostril opening in one bolus via a micropipettor.

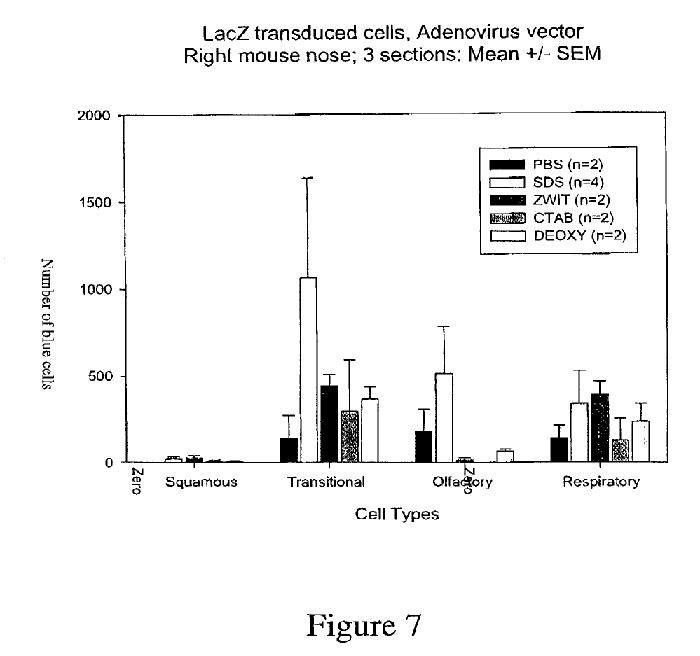

The mouse was set aside for one hour; re-anaesthetised as needed, 1 hour later 20 μl of viral vector was instilled into the same nostril. The mouse was allowed to recover and returned to caging. Three days later the mice were killed with CO₂ excess, head removed and the nasal airways were flushed with fixative. These were taken through standard “X-gal” processing to reveal blue stained cells that represent LacZ gene transfer. Cells were processing to wax sections and stained with Safranin-O or haemotoxylin/oesin. Stained cells present along the perimeter of 3 standard cross-sections in the treated nasal airway were counted. Counts were separately made for each type of epithelium in the nasal airway perimeters (respiratory, squamous, olfactory, transitional). Data was then graphed as shown in FIG. 7. It can be seen that there is a considerable increase in numbers of transductants using the detergents over the control PBS in the transitional cells, and also for respiratory cells in the latter particularly SDS, Zwit and Deoxy. It is considered that the respiratory epitheum is most likely to be important where the invention is to be used to ameliorate a respiratory condition.

### EXAMPLE 3

Assessing Damage of Epithelia on Treatment of Penetrating Agent

The method of choice for assessing an appropriate level of damage is by the measurement of transepithelial potential difference (TDP) (Middleton et al., 1994; Knowles et al., 1995; Lee et al., 1999). In general, damage to epithelium with penetrating agents cause at least opening of the tight junctions and this causes the resident negative TPD on the airway surface to go toward zero. If there is exfoliation (that is substantial damage) then the TPD goes to zero quickly and takes several days to return to normal because the epithelia cells have been stripped off and have to regenerate.

Measurement of TPD was performed under zero-chloride perfusion to give a larger baseline TPD value from which to measure change. Under these conditions the values are typically around −20 mV compared to about −5 mV if normal perfusion saline is used. Mice are anaesthetised, TPD cannula is inserted, then the detergent dose is instilled in the same nostril, the effect is then followed in this anterior part of the nose. The methods used for measuring TDP are largely as described in Parsons et al., 1998. For method for use in human see Middleton et al., 1994 and Knowles et al., 1995.

Experiment 1.

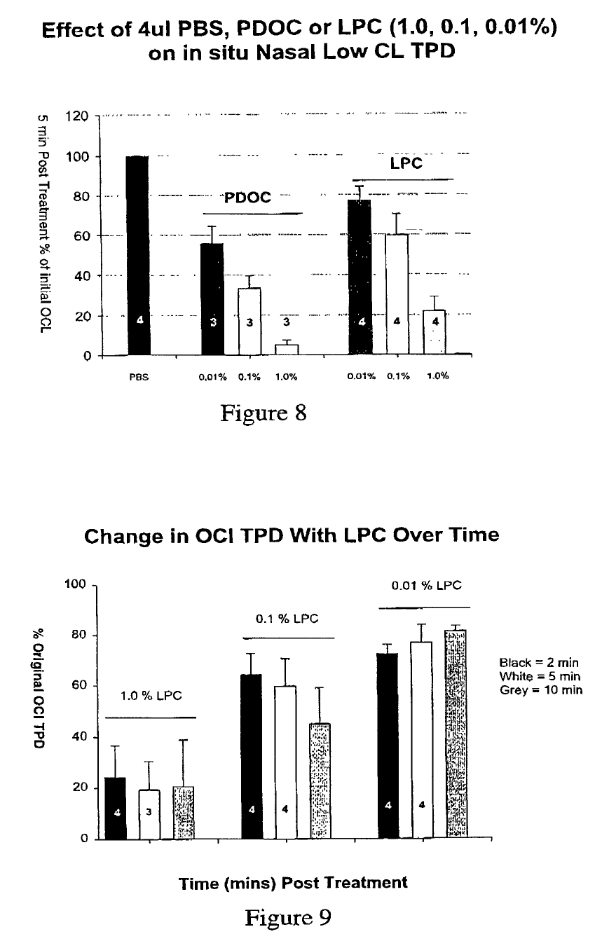

The dosage of detergent used is 4 μl of PBS, PDOC of LPC and is in line with methods used in the above examples. The solutions of detergent use are 0.01, 0.1 and 1.0%. The TDP value was determined 5 minutes after administration of the detergent. The values are presented graphically in FIG. 8. It can be seen that PDOC has a greater effect on TDP value than LPC and is thus more damaging. A score of 100% represents the pretest TDP value. It is suggested by the present inventos that a score of about 70% of the pretest TDP may represent adequate dmage to the surface epitheilia layer for transduction to occure. A TDP value of less than about 60% is though to be preferable.

Experiment 2.

The same doses of LPC were used as for experiment 1 but TDP was measured at 2, 5 and 10 minutes after administration. The data are presented graphically in FIG. 9. After 10 minutes the 0.01% LPC was not significantly different to pretest TPD. Thus should 0.01% LPC be used for treatment of the lung then lentivirus should be delivered within 10 minutes.

Experiment 3.

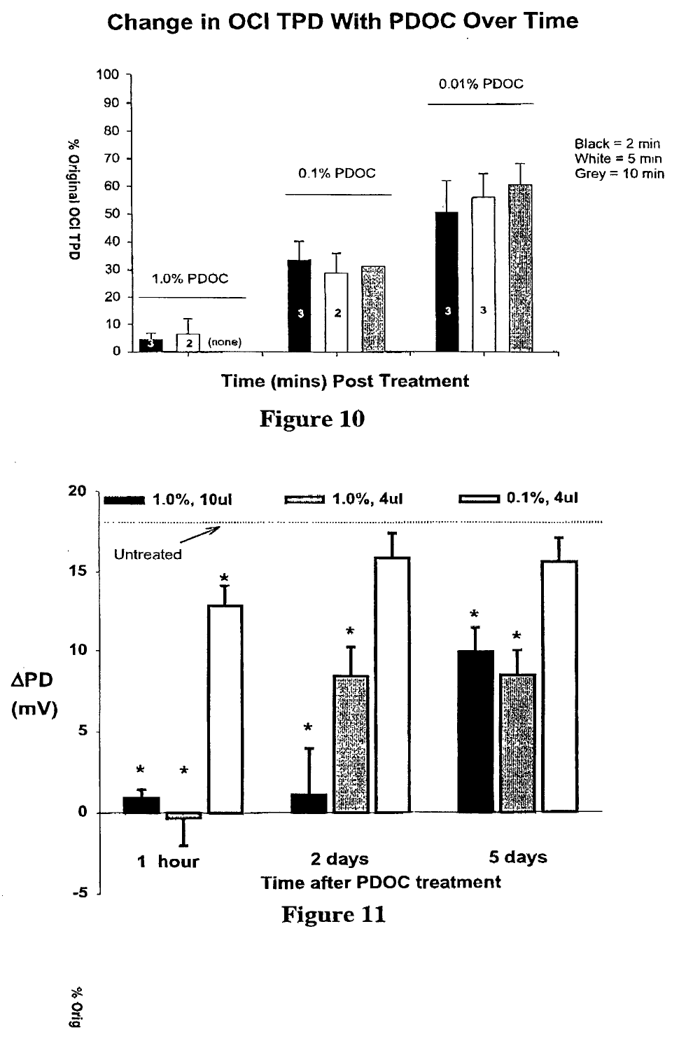

This uses that same doses of PDOC as in experiment 1 and as in experiment 2 TDP was measured at 2, 5 and 10 minutes after administration. The data from this experiment are presented graphically in FIG. 10.

Experiment 4

TDP was measured over a longer time course using PDOC as the penetrating agent. The doses used in this experiment were (per mouse) 10 μl of 1.0% PDOC, 4 μl of 1.0% PDOC and 4 μl of 0.1% PDOC. The data from this experiment are presented in FIG. 11. It can be seen that the recovery of TDP function is extended with the first two doses but rapidly recovers where 4 ml of 0.1% PDOC is used. A recovery of TDP function within about 1 day is considered to be transient.

Experiment 5

The effect of detergents (polidocanol and lysophospatidylcholine) on the level of viral gene transfer were firstly evaluated in vitro.

Initial experiments made use of the AdV5CMVLacZ vector (a generous gift from Dr. R. C. Boucher). The concentration of polidocanol and its contact with the cells that did not cause significant (ie >5%) cell mortality was chosen as the optimal treatment dose. This dose of polidocanol was assessed on polarised cells (MDCK) and also non-polarised cells (A549). The AdV vector was added at 10, 30 and 60 mins following treatment. For the non-polarised cells there was no apparent difference in the level of gene transfer between the three different vector instillation times. However, for the polarised cells gene transfer was significantly improved (P<0.05, ANOVA, SNK) when the vector was added one hour after treatment, compared to the shorter time points. When the experiments were repeated using the LV vector we found the same effect.

### EXAMPLE 4

Construction of Lentiviral Particle

Replication-defective retroviral vectors are advantageous for gene therapy applications where stable genetic modification of the target cell is the desired outcome as the chromosomally integrated proviral form is the endpoint of the (replication-defective) retroviral life cycle. Within the retroviruses, at least in terms of developing useful gene transfer vectors, lentiviruses such as human immunodeficiency virus type 1 (HIV-1) have the additional advantage of being able to infect non-dividing cell populations (Lewis et al., 1992) even though not all such cell populations appear equally susceptible to infection (Korin and Zack, 1998; Sutton et al., 1999; Chinnasamy et al., 2000; Park et al., 2000). This has led to the development of vector systems from a number of lentiviruses including HIV-1 (Naldini et al., 1996), HIV-2 (Sadaie et al., 1998) and feline immunodeficiency virus (Poeschla et al., 1998). Initial testing of these vectors has demonstrated that they appear well-suited to the efficient transduction of different tissues and cells of interest in various gene therapy strategies including neurones (Blomer et al., 1997), retinal cells (Miyoshi et al., 1997) and haematopoietic cells (Sutton et al., 1998) as well as liver and muscle cells (Kafri et al., 1997). The pathogenic nature of HIV-1 raises some concerns about safety, especially when using transient expression systems for virus production where recombination events that potentially lead to the production of replication-competent viruses would appear to be more likely than in stable packaging/producer cell lines.

The main safety criterion for retroviral gene vector systems of any sort is the absence of replication-competent virus. Replication-competent viruses can be generated by recombination of the constituents of the virus production system (i.e. vector and packaging constructs) with each other or with endogenous viral sequences in the cell lines used to generate virus. The probability of such recombination occurring can be minimised by separating the packaging (viral trans) functions onto several different plasmids and by reducing homology between the different constituents of the vector production system as far as possible. The power of designing virus production systems in this way has been well demonstrated over the years during the development of vector/packaging systems from oncogenic viruses such as the Murine Leukemia Viruses (Cosset et al., 1995; Rigg et al., 1996).

The safety of HIV-1 derived vectors has been enhanced by improvements to both vector and helper plasmid design. Considerable effort has been directed at minimising the amount of HIV-1 sequence in the vector, both by using heterologous sequences to substitute for HIV-1 promoter and polyadenylation signals and by making “self inactivating” HIV-1 vectors (Dull et al., 1998; Miyoshi et al., 1998; Cui et al., 1999; Iwakuma et al., 1999).

To date HIV-1 helper systems have mostly been constructed by successive alteration of proviral clones to prevent autonomous replication. This has included the use of heterologous promoter and polyadenylation sequences, deletion of the packaging signal and other untranslated sequences, and deletion of non-essential coding sequences such as env and some or all of the minor proteins (Naidini et al., 1996; Zufferey et al., 1997; Kim et al., 1998; Chang et al., 1999; Srinivasakumar and Schuening, 1999). In some instances a minimal gagpol expression plasmid has been used with Rev, the only other essential HIV-1 protein, provided from a separate plasmid (Dull et al., 1998). More recently a packaging system that utilises separate constructs for expression of Gag-Pro (Protease) and Vpr-RT-IN (Integrase) polyproteins has been described (Wu et al., 2000). In an attempt to reduce the risk of episomal recombination events that may occur in transient virus production systems, stable packaging cell lines have also been developed for HIV-1 derived vectors (Klages et al., 2000; Xu et al., 2001).

In order to help achieve the aim of a safe and effective HIV-1 based vector system we have expressed all the relevant HIV-1 reading frames (gagpol, tat (exon 1), rev, vif, vpu, vpr, and nej) from separate plasmid constructs. In each case a minimal reading frame has been linked to heterologous transcriptional regulatory sequences. The gagpol sequence was codon-optimised for expression in mammalian cells allowing efficient Rev/RRE independent expression. Similar codon-optimised gagpol reading frames have also recently been described by Kotsopoulou et al. (2000) and Wagner et al. (2000). In addition, we show that the Gag and GagPol polyproteins can be efficiently expressed at high levels from separate, codon-optimised, expression constructs allowing the removal of the HIV-1 gagpol translational frameshift sequence from the virus production system. We show that these plasmids provide the basis for the production of recombinant HIV-1 vectors in transient expression systems with improved safety as measured by the frequency of coincident transfer of biologically active sequences encoding Gag/GagPol (ie sequences equivalent to the gagpol gene) to transduced cells.

Material and Methods

Materials

The pYU-2 plasmid contains a proviral clone of HIV-1 YU-2 (Li et al., 1992; Genbank accession number M93258) in pTZ19U. The pHCMV-G plasmid expresses the vesicular stomatis virus G protein (Yee et al., 1994) and the HIV-1 packaging plasmid pCMVΔRnr expresses all HIV-1 trans functions with the exception of Vpr, Vif and Env (Kafri et al., 1997). The Rev expression plasmid pCMV-rev (Lewis et al., 1990) was obtained from the NIH AIDS research and reference reagent program (catalogue number 1443). The HIV-1 vectors pB1HIVext5SV40EYFPppt+RRELTR (FIG. 1) and pB1HIVext5SV40Neoppt+RRELTR transduce the enhanced yellow fluorescent protein (EYFP, Clontech) and neomycin resistance (Neo^{r}), respectively, under the transcriptional control of the SV40 early promoter. While these are not fully optimised vectors they are packaged by pCMVΔRnr (Kafri et al., 1997) with relatively high efficiency (e.g. approximately 2000 NIH3T3 transducing units per ng of p24 for pB1HIVext5SV40EYFPppt+RRELTR). Both these constructs are self-inactivating due to the replacement of U3 sequences between the EcoRV and PvuII sites in the 3′ long terminal repeat (LTR) by the YU-2 RRE. A detailed description of the vector construction will be published elsewhere (manuscript in preparation). Antiserum to Nef (catalogue number 2949), Vif (catalogue number 2221), Vpr (catalogue number 3252) and Vpu (catalogue number 969) were obtained from the NIH AIDS research and reference reagent program. Cloning vectors used were pBluescript II SK (+) (Stratagene) and pcDNA3.1 (Invitrogen). Oligonucleotides (50 nmole scale, de-salted) were purchased from Gibco-BRL. DNA cloning and manipulation were done using standard procedures (Ausubel et al., 1989).

Isolation of Sequence Elements

Isolation of specific sequence elements by PCR was performed using a proof-reading polymerase mix (Pwo or Expand High Fidelity, Boehringer Mannheim) utilising primers incorporating restriction enzyme recognition sequences to facilitate further manipulation and the minimum number of PCR cycles required (generally between 5 and 10) to produce enough material for cloning purposes. In each case the PCR product was purified using a Qiagen or Geneworks spin column, restricted with the appropriate enzymes, gel-purified, cloned and sequenced.

The YU-2 RRE was isolated from pYU2 by PCR as a 5′ XbaI/3′ ApaI fragment (YU-2 bases 7734-7974) using primers incorporating the relevant restriction enzyme sites. The primers used were RRE5′XBA, (5′ GGGCCTCTAGAGCTTTGTTCCTTGGGTTCTTG 3′), and RRE3′APA (5′ GGGTCGGGCCCAAATCCCTAGGAGCTGTTG 3′). The RRE was then cloned between the XbaI and ApaI sites of pcDNA3.1 to give pcDNA3rre.

The vif, vpu, nef, tat (exon 1) and vpr reading frames were isolated by PCR using primers encompassing the ATG initiation codon and the relevant stop codon for each reading frame with no extraneous (i.e. 5′ or 3′ of these codons, respectively) HIV-1 sequence included.

Restriction enzyme sites that facilitated subsequent cloning steps were included in each primer. In addition, a Kozak translation initiation consensus sequence (GCCACC) was added upstream of the ATG initiation codon. The primer pairs used were (i) for cloning the vif coding sequence (YU-2 bases 5039-5617) as an EcoRI/BamHI fragment, vifF (5′ CGGGAATTCGCCACCATGGAAAACAGATGGCAGGTGATG 3′) and vifR (5′ ACGCGGATCCCTAGTGTCCATTCATTGTGCGGCT 3′), (ii) for cloning the vpu coding sequence (YU-2 bases 6062-6307 with base 6062 changed from a C in the YU-2 sequence to an A) as a NcoI/NotI fragment, vpuF (5′ GCGCATGCCATGGCCACCATGCAATCTTTACAAGTATTAGCA 3′) and vpuR (5′ ATAAGAATGCGGCCGCTACAGATCATCAACATCCCAAGG 3′), (iii) for cloning the vpr coding sequence (YU-2 bases 5557-5850) as a PstI/BamHI fragment, vprF (5′ CGGCTGCAGGCCACCATGGAACAAGCCCCAGAAGACCAA 3′) and vprR (5′ ACGCGGATCCTTAGGATCTACTGGCTCCATTTCT 3′), (iv) for cloning the nef coding sequence (YU-2 bases 8758-9402) as a NcoI/NotI fragment, nefF (5′ GCGCATGCCATGGCCACCATGGGTGGCAAGTGGTCAAAACGT 3′) and nefR (5′ ATAAGAATGCGGCCGCTCAGTTCTTGTAGTACTCCGGATG 3′) and (v) for cloning the first exon of the tat reading frame (YU-2 bases 5831-6049) as an EcoRI/XbaI fragment, tatF (5′ ACGCTGAATTCGCCACCATGGAGCCAGTAGATCCTAACCTA 3′) and tatR (5′ CTATGCTCTAGATTACTGCTTTGATAGAGAACTTTG 3′). Each PCR product was then cloned via the relevant restriction enzyme sites into the expression vector pcDNA3.1 to give pcDNA3Vif, pcDNA3Vpu, pcDNA3Vpr, pcDNA3Nef and pcDNA3Tat, respectively.

The Mason-Pfizer monkey virus constitutive transport element (MPMV-CTE) (nucleotides 7386-7554, Genbank accession number AF033815, Pasquinelli et al., 1997) flanked by EcoRV restriction enzyme sites was synthesised by controlled annealing of a series of overlapping oligonucleotides and cloned into the EcoRV site of pBluescript II SK(+). DNA sequencing was used to identify a clone containing the correct sequence and the MPMV-CTE was then isolated as an EcoRV fragment and subcloned into the (blunt-ended) ApaI site of pcDNA3.1 to give pcDNA3cte.

Synthesis of Codon-Optimised Reading Frames for YU-2 Gag/GagPol

The codon-optimised gagpol gene sequence was made by assembling overlapping 50mer oligonucleotides with a PCR-based approach essentially as described by Stemmer et al. (1995). The sequence was assembled as 4 sub-fragments of 797, 1085, 1108 and 1315 bases in length making use of naturally occurring restriction enzyme sites within the sequence. Multiple clones of each fragment were isolated and fully sequenced to identify clones containing either error-free sequence or pairs of clones that could be recombined by PCR to give the correct sequence. The complete sequence (gagpolml, Genbank accession number AF287352) was then assembled as a 5′ HindIII/3′ XbaI sequence in pBluescript II SK(+) (pBlgagpolml) using standard subcloning procedures. Details of the assembly procedure are available on request. The gagpolml coding sequence was then subcloned as a HindIII/XbaI fragment into the expression vector pcDNA3.1. A variant of this sequence, gagpolfusionml (Genbank accession number AF287353), in which the frameshift and region of overlap between the reading frames encoding Gag and Pol were replaced by codon-optimised sequence encoding the GagPol polyprotein, was then made. This construct is therefore designed to express only the GagPol polyprotein and utilises entirely codon-optimised sequence.

Separate codon-optimised reading frames for Gag and Pol were constructed as follows. For Gag the frameshift region and Pol reading frame in pBlgagpolml were replaced with codon-optimised sequence to complete the coding sequence for Gag (pBlgagml) flanked by 5′ HindIII and 3′ XbaI restriction enzyme sites. For Pol (pBlpolml) the Gag coding sequence up to the first codon of the reading frame for Pol in pBlgagpolml was replaced with a sequence encompassing a HindIII site, a short non-coding region and an ATG initiation codon in-frame with the Pol reading frame. The resulting sequences have been deposited in Genbank (accession numbers AF287354 and AF287355, respectively).

Expression constructs for the gagpolml, gagml and gagpolfusionml sequences that contained the HIV-1 RRE or the MPMV-CTE in the 3′ non-coding sequence were made by re-cloning the relevant sequences into pcDNA3rre or pcDNA3cte (see above) to give pcDNA3gagpolmlrre, pcDNA3gagmlrre, pcDNAgagmlcte, pcDNAgagpolfusionmlrre and pcDNAgagpolfusionmlcte.

Cell Culture and Transfection

All cells were grown in DMEM/10% (v/v) FCS and sub-cultured by treatment with trypsin/versene solution. Twenty-four hours prior to transfection 293T (ATCC SD3515) cells were plated at 10⁶ cells per well in 6 well plates (35 mm diameter wells) and subsequently transfected by calcium phosphate co-precipitation (Jordan et al., 1996). Unless otherwise stated a total of 3 μg of DNA per well was used. Where necessary, carrier DNA was added to keep the total mass of DNA per transfection constant. Twenty four hours after addition of the calcium phosphate co-precipitate the medium was aspirated and replaced with 2 ml of fresh pre-warmed medium. Conditioned medium was collected 48 hours later, sterile (0.2 μm) filtered and analysed. All direct comparisons were done within individual experiments to minimise experimental variation. For analysis of expression of the minor proteins, Cos-1 cells (ATCC CRL 1650) (100 mm dish) were transfected with 10 μg of plasmid DNA using Lipofectamine (Boehringer Mannheim) and cell lysates harvested for analysis 72 hours later.

For determination of virus titre NIH3T3 cells (ATCC CRL 1658) were plated at 1.2×10⁶ cells/well in 6-well plates. Six hours later the medium was aspirated and virus supernatant (diluted with fresh medium to a total volume of 2 ml) was added in the presence of 4 μg/ml polybrene. Twenty-four hours later the medium was removed and replaced with fresh medium, and after a further 24 hours the cells were subcultured 1:3. Another 24 hours later the cells were harvested and assessed for expression of EYFP by FACScan analysis. In some experiments transduced cells were sub-cultured for up to 10 days. Transduction, as measured by EYFP expression in selected samples, was stable between 72 hours and 10 days after transduction indicating that pseudo-transduction of EYFP was not occurring.

Assay for Transfer of Sequences Encoding Gag and GagPol

High titre stocks of virus produced using either the pCMVΔRnr, the pcDNA3gagpolml or the pcDNA3gagml/pcDNA3gagpolfusionml helper systems were prepared by a combination of ultrafiltration (Amicon stirred cell with ZM500 membrane) and ultracentrifugation and their titre determined on NIH3T3 cells as described above. 293T cells were plated at 1.5×10⁶ cells per well in 6 well dishes and 6 hours later transduced with virus in the presence of 4 μg/ml polybrene in a total volume of 2 ml. After transduction the cells were expanded and, for each assay, 2×10⁶ cells were plated in a 60-mm dish. Untransduced 293T cells were used as a control. Twenty-four hours after plating, the cells were transfected with a mixture of plasmids encoding either, (i) gagpolfusionml, VSV-G, Tat, Rev and pHIVext5SV40Neoppt+RRELTR (FIG. 1) or, (ii) VSV-G, Tat, Rev and pHIVext5SV40Neoppt+RRELTR. Conditioned medium was then collected, 0.2 μm filtered and assayed on A549 cells (ATCC CCL-185, these were used as they give approximately ten to twenty times higher titres than NIH3T3 cells) for Neo^{r} transducing units as follows. A549 cells were plated at two million cells per 60 mm dish and 5 hours later transduced, in the presence of 4 μg/ml polybrene, with the entire volume of conditioned medium collected from the transduced/transfected 293T cells. Twenty-four hours later the cells were subcultured into 3×100 mm dishes and selected with 1 mg/ml (total) G418 for 2 weeks after which time G418 resistant colonies were enumerated. In the first of these assays (transfection with gagpolfusionml-containing mix) production of Neo^{r} transducing virus particles will depend on the transfer of biologically active sequences encoding Gag to the 293T cells via the virus stock being assayed. We have called this the “gag transfer” assay. In the second instance, production of Neo^{r} transducing virus particles will depend on transfer of biologically active sequences capable of substituting for the HIV-1 gagpol gene sequence (i.e. capable of expressing both the Gag and GagPol polyproteins). We have called this assay the “gagpol transfer” assay.

FACScan Analysis

Cells for analysis were harvested by trypsinisation, washed in PBS and then resuspended in PBS/10 μg/ml propidium iodide. Cells were analysed for EYFP and propidium iodide fluorescence and the results analysed using Cellquest software (version 3.0.1 f, Becton Dickinson). The enumeration of EYFP positive cells was limited to the live cell population as defined by low propidium iodide fluorescence and by forward/side scatter plots. Histogram markers for EYFP positive cells were used such that untransduced NIH3T3 cells gave a false positive rate of 0.26%±0.10% (range 0.16-0.43, n=10). For all experiments a false background rate of 1% was then assumed and deducted from the experimental value. Virus titre was calculated by multiplying the percentage of positive cells by the number of NIH3T3 cells used (1.2×10⁶) and adjusting for the volume of virus added.

HIV-1 p24 and RT Assays

HIV-1 p24 and RT measurements were made using commercial ELISA kits from NEN Life Sciences and Boehringer Mannheim, respectively.

Western Blot Analysis

For analysis of the HIV-1 minor proteins Vif, Vpr, Vpu and Nef, transfected Cos-1 cells were harvested and cell lysates were prepared by 5 cycles of freeze/thaw in 150 mM NaCl, 1% (v/v) NP40, 0.5% (w/v) DOC, 0.1% (w/v) SDS, 50 mM Tris-HCl, pH 8.0. Lysates were then clarified by microcentrifugation and 10 μg of total protein electrophoresed on a 15% denaturing SDS-PAGE minigel and then transferred onto a nitrocellulose membrane. The membrane was then hybridised with the relevant antibody at dilutions suggested by the supplier and signals detected with an alkaline phosphatase-conjugated secondary antibody and BCIP/NBT (Sigma). For analysis of Gag/GagPol, lysates were prepared from transfected 293T cells as described above (for Cos-1) and 100 μg electrophoresed on a 12.5% denaturing SDS-PAGE gel and transferred onto a nitrocellulose membrane. The membrane was then hybridised with heat inactivated HIV-1 positive human serum and signals detected with a horseradish peroxidase-conjugated secondary antibody and 4-chloro-1-napthol.

Statistical Analysis

Unless otherwise indicated results are given as mean±standard deviation. Where n=2 the mean and individual values are given. Where appropriate a two-tailed Students t-test was used to compare two sets of data. For multiple comparisons of data a one way ANOVA was performed using the Student-Newman-Keuls (SNK) method. Results were considered significantly different at p<0.05.

Results

Expression of HIV-1 YU-2 Gagpol Gene Products from a Single Codon-Optimised Transcriptional Unit

In order to produce a codon-optimised gagpol gene sequence the coding sequence of the YU-2 gagpol gene was back-translated using optimal codon usage for mammalian gene expression (Australian National Genome Information Service (ANGIS) mam_h.cod). The only sequence not codon-optimised was from base 2060 to 2294 of the YU-2 sequence. This region encompasses the translational frameshift signal for expression of the GagPol polyprotein and the region of overlap between the gag (Gag amino acids 425 to 500) and pol (Pol amino acids 1 to 69) reading frames. A HindIII site and a short 5′ non-translated sequence containing a Kozak consensus sequence were added at the 5′ end of the Gag coding sequence and an XbaI site at the 3′ end. This sequence was cloned between the HindIII and XbaI sites of pcDNA3.1 to give pcDNA3gagpolml (FIG. 13). A derivative of this, pcDNA3gagpolmlrre, which contained the YU-2 RRE in the 3′ non-coding sequence, was also made.

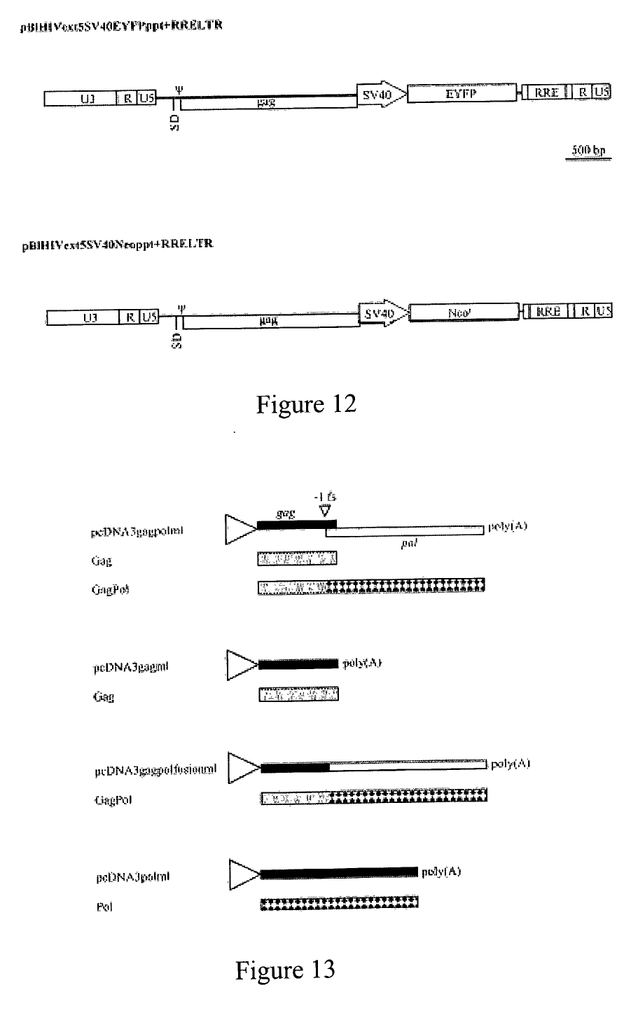

A constant molar quantity (100 fmoles) of pcDNA3gagpolml and pcDNA3gagpolmlrre (653 or 671 ng, respectively) was co-transfected with pCMV-rev (0.5 μg), pcDNA3Tat (0.5 μg), pHCMV-G (0.5 μg) and pB1HIVext5SV40EYFPppt+RRELTR (1 μg) (FIG. 12 and see Materials and methods) and the resulting samples assayed for p24, RT and virus titre as described in Material and methods. The pCMVΔRnr construct (Kafri et al., 1997, (100 mmoles, 850 ng)) was used as a comparative control (replacing the gagpol, rev and tat constructs).

The results of testing the codon-optimised gagpol gene sequence (pcDNA3gagpolml) demonstrated that it supports efficient p24 expression (477 ng/ml) and virus production (a titre of 2.4×10⁵ EYFP TU/ml) (Table 1). These figures represent 80% and 36% of the corresponding values obtained with the pCMVΔRnr proviral helper construct. In contrast expression of RT from pcDNA3gagpolml was 147% of the level from pCMVΔRnr. The addition of the RRE to the codon-optimised construct (pcDNA3gagpolmlrre) resulted in a marginal decrease in p24 expression and virus titre (Table 1).

Expression of HIV-1 YU-2 Gagpol Gene Products from Separate Transcriptional Units

Separate codon-optimised expression constructs for the Gag (pcDNA3gagml), Pol (pcDNA3polml) and GagPol (pcDNA3gagpolfusionml) polyproteins (FIG. 13) were made as described in Materials and methods. Derivatives of pcDNA3gagpolml and pcDNA3gagpolfusionml with the YU-2 RRE or MPMV-CTE in the 3′ non-coding region were also made (pcDNA3gagmlrre, pcDNA3gagmlcte, pcDNA3gagpolfusionmlrre and pcDNA3gagpolfusionmlcte, respectively).

Again a constant molar amount (100 fmoles) of the Gag polyprotein expression constructs (455 or 462 ng depending on the construct) and the stated amount (see Tables 2 and 3) of the GagPol polyprotein or Pol polyprotein plasmid) were co-transfected with pCMV-rev (0.5 μg), pcDNA3Tat (0.5 μg), pHCMV-G (0.5 μg) and pB1HIVext5SV40EYFPppt+RRELTR (1 μg).

The resulting samples were assayed for p24, RT and virus titre as described in Materials and methods.

The results of these experiments showed that none of these expression constructs can on their own support the production of detectable virus titres (Tables 2 and 3). The combination of the pcDNA3gagml and pcDNA3polml constructs was also unable to support the production of detectable virus titres although they were capable of expressing p24 and RT activity, respectively (Table 3). In contrast, the combination of pcDNA3gagml and pcDNA3gagpolfusionml constructs was able to substitute for the normal expression of these two proteins from a single transcriptional unit as occurs via frameshifting in both the native gagpol gene and the pcDNA3gagpolml codon-optimised construct. This was demonstrated both by the expression of the two polyproteins, as assessed by p24 and RT assays, and by the production of virus particles as measured by transduction of EYFP expression into NIH3T3 cells (Table 2). Both p24 and RT were expressed at levels comparable to those obtained from pCMVΔRnr (500 to 600 ng/ml and 1 to 2.5 ng/ml, respectively). However, the corresponding virus titre was approximately six-fold lower, around 100 EYFP TU/ml, versus 6×10⁵ EYFP TU/ml for pCMVΔRnr. Addition of the RRE or the MPMV-CTE into these constructs had no discernible effect on the level of expression of p24 or on virus titre The molar ratio of the pcDNA3gagpolml and pcDNA3gagpolfusionml expression plasmids (+/−RRE and CTE) was varied between 20:1 and 2.5:1 (a molar ratio of 5:1 is depicted in Table 2) while keeping the level of pcDNA3gagml constant (at 100 fmoles), and had little effect on the levels of expression of p24 and RT, or on virus titre (data not shown).

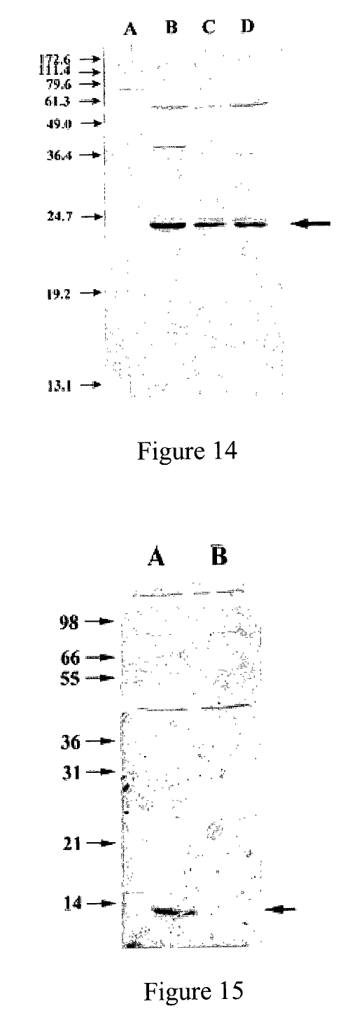

Western blot analysis of cell lysates prepared from 293T cells transfected with either pCMVΔRnr, pcDNA3gagpolml or a mixture of pcDNA3gagml and pcDNA3gagpolfusionml and probed with serum from a HIV-1 positive patient clearly indicated the presence of correctly processed p24 in each sample (FIG. 14). No obvious differences between these samples in the processing of Gag were apparent from this analysis.

Expression of, and Requirement for, the HIV-1 Regulatory Proteins Tat and Rev

The first exon of the YU-2 tat gene was isolated by PCR and cloned into pcDNA3.1 as described in Materials and methods to give pcDNA3Tat. To test the effect of Tat expression on virus titre pcDNA3gagpolml (653 ng, 100 fmoles), pB1HIVext5SV40EYFPppt+RRELTR (1 μg), pHCMV-G (0.5 μg) and pCMV-rev (0.5 μg) were co-transfected with and without pcDNA3Tat (0.5 μg). The results (Table 4) show that addition of Tat increases virus titre almost three-fold (p<0.001).

Rev was expressed from a previously described construct, pCMV-rev (Lewis et al., 1990). To test the requirement for Rev in virus production pcDNA3gagpolml (653 ng, 100 fmoles), pB1HIVext5SV40EYFPppt+RRELTR (1 μg), pcDNA3Tat (0.5 μg) and pHCMV-G (0.5 μg) were co-transfected with and without pCMV-rev (0.5 μg). The results (Table 5) clearly demonstrate that Rev is absolutely required for production of detectable virus titres.

Expression of the HIV-1 Minor Proteins Vif, Vpu, Vpr, Nef

Individual expression constructs for each of the YU-2 minor proteins were made using minimal HIV-1 sequence as described in Materials and methods. The expression constructs for Vif, Vpu, Vpr and Nef were assessed by Western blot analysis after transient expression in Cos-1 cells using antibodies available from the AIDS reagent program (see Materials and methods). We were only able to demonstrate expression of Vpr (FIG. 15) by this method, partly due to the high background observed with the other antibodies. To assess the effect of the expression of minor proteins on virus titre pB1HIVext5SV40EYFPppt+RRELTR (0.4 μg), pcDNA3gagpolml (0.4 μg), pcDNA3Tat (0.4 μg), pCMV-rev (0.4 μg) and pCMV-G (0.2 μg) were co-transfected with or without all 4 minor protein expression constructs (0.3 μg of each). The results (Table 6) show that addition of the minor proteins caused a small but significant (26%) increase in virus titre as assayed on NIH3T3 cells (p<0.05). In terms of titre per ng of p24 the effect of the addition of the minor proteins is greater, resulting in nearly a two-fold increase (p<0.001).

Transfer of Gag and Gagpol Sequences

Virus was prepared using either the pCMVΔRnr, pcDNA3gagpolml or pcDNA3gagml/pcDNA3gagpolfusionml helper systems and concentrated to high titres as described in Materials and methods. Triplicate samples of each virus preparation were then tested in the “gag” and “gagpol” transfer assays (see Materials and methods). The results of these assays are shown in Table 7. The untransduced 293T control indicated the presence of a low background in both assays. The pCMVΔRnr virus generated a large readout in each assay. With the virus produced using the pcDNA3gagpolml system a readout was detected in both the gag and gagpol transfer assays although at a significantly reduced level (approximately 1%) compared with the pCMVΔRnr virus (p<0.05). With virus produced using the pcDNA3gagml/pcDNA3gagpolfusionml system only the gag transfer assay gave a readout above the observed background, again at a significantly reduced level (approximately 8%) compared with the virus made using pCMVΔRnr (p<0.05). The results of the gag assay for the pcDNA3gagpolml virus and the pcDNA3gagml/pcDNA3gagpolfusionml virus were not significantly different (p>0.05) while the results for the same comparison in the gagpol assay were significantly different (p<0.05).

Transduction of Cells at a High Multiplicity of Infection

As the virus produced with the pcDNA3gagpolml and pcDNA3gagml/pcDNA3gagpolfusionml has a lower titre per ng of p24 than the virus prepared with pCMVΔRnr, we investigated whether this could potentially interfere with transduction at high multiplicities of infection (moi). Accordingly, A549 cells were transduced at an estimated moi of 0.1, 1 and 10 using virus prepared with either pCMVΔRnr or pcDNA3gagpolml. The respective titres of these virus preparations were 4516 and 1876 NIH3T3 transducing units/ng of p24. The results (Table 8) show that both viruses are able to transduce cells effectively at high moi (>95% transduction for both) but that at an moi of 1 the virus prepared with pcDNA3gagpolml is somewhat less effective (52% versus 67% EYFP positive cells) than that prepared with pCMVΔRnr. However, analysis of the mean fluorescence of the cells transduced at an moi of ten suggests that the real rate of transduction is at least three fold lower with the virus prepared using pcDNA3gagpolml than with the virus made using pCMVΔRnr.

Discussion

Helper Plasmids with a Codon Optimised Gagpol Gene

We have taken as our starting point in the development of an HIV-1 gene transfer vector system the complete disassembly of the HIV-1 YU-2 trans functions (with the exception of Env, which is substituted with the VSV-G protein). The YU-2 strain was chosen as a fully replication-competent, non-cell culture-adapted strain capable of infecting macrophages. Each of the relevant reading frames has been isolated as a minimal or near minimal transcriptional unit and cloned into separate expression constructs. In addition the gagpol gene was codon-optimised for high-level expression in mammalian cells. The resulting construct, pcDNA3gagpolml, expressed p24 at approximately 80% of the level resulting from the proviral-type helper construct pCMVΔRnr. Codon-optimisation also makes expression independent of Rev/RRE despite the requirement to leave the frameshift signal and the region of overlap between the gag and pol reading frames unaltered. Although the cis sequences that prevent efficient nuclear export of the native gagpol transcript have not been completely defined, these are presumably destroyed by codon-optimisation. For example, codon-optimisation removes all the AUUUA RNA destabilisation cis sequence elements in the gagpol sequence and this is likely to be one mechanism through which expression is enhanced. Kotsopoulou et al. (2000) and Wagner et al. (2000) have described similar codon-optimised reading frames for the HIV-1 gagpol gene. Kotsopoulou et al. (2000) showed that codon-optimisation results primarily in increased mRNA levels. Codon-optimisation itself would also be expected to directly enhance the translational efficiency of the transcript.

A second important consequence of codon-optimisation is that homology with the native gagpol gene sequence is reduced to 75% overall thus reducing in turn the probability of recombination between vectors containing gag sequences, or HIV-1 itself, and gagpol helper constructs. In addition, the removal of all 5′ non-coding sequence also reduces homology with the vector construct. This effect is clearly shown by the results of our gag and gagpol transfer assays when comparing virus made with pCMVΔRnr and pcDNA3gagpolml which show that the use of the codon-optimised construct reduces detectable sequence transfer approximately 100 fold. In addition codon-optimisation destroys the vif reading frame overlapping the 3′ end of the native pol coding sequence. However, although the levels of p24 and RT expression from pcDNA3gagpolml are comparable to pCMVΔRnr (being 80% and 150%, respectively, of the values for pCMVΔRnr) the resulting virus titre is only 36% of that obtained with pCMVΔRnr. One possible explanation for this observation is that the expression of Tat and Rev in our system may not be optimal as their expression is no longer coordinately regulated with expression of GagPol. Furthermore, pCMVΔRnr expresses Nef and Vpu and our results suggest that the minor proteins can influence virus titre (Table 4 and see below). It is also possible that the native sequence contains signals to co-localise translation of gagpol with the genomic RNA to be packaged so increasing the efficiency of virus production. There is some evidence to suggest that virion assembly is localised within the cell (Rhee and Hunter, 1991). We are currently investigating these issues further.

Separation of the Gagpol Transcription Unit

Having achieved the efficient Rev/RRE independent expression from the gagpol gene we then extended the idea of maximal separation of the trans functions by attempting to express the two polyproteins encoded by the gagpol transcription unit via two separate plasmids, again using codon-optimised coding sequences. Not surprisingly, given the coordinated and orderly processing of the Gag and GagPol polyproteins and the functional linkage of these processes to their incorporation into the virion, the use of separate reading frames for Gag and Pol did not result in detectable virus titre although both p24 and RT were detected in the medium. It is well known that the expression of Gag, the major structural protein of the virus core, will result in the formation of virus-like particles (Shioda and Shibuta, 1990). However, it is thought that the Gag portion of the GagPol polyprotein is required for its incorporation into virus particles, making the presence of large amounts of RT in the medium harder to explain. The observation of similar levels of extracellular RT with the Pol plasmid alone suggests that the enzyme may not necessarily be associated with virions.

In contrast, the use of separate reading frames for the Gag (pcDNA3gagml) and GagPol (pcDNA3gagpolfusionml) polyproteins in combination did result in the efficient synthesis of p24 and RT, and in high virus titres. Neither construct on its own supports the generation of a detectable virus titre because, in each case, essential viral coding sequence is absent. For the gagml construct this is the entire pol gene, and for the gagpolfusionml construct the sequence coding for the p6 protein, which would normally be encoded in the region of overlap between the Gag and Pol reading frames. However, the virus titre for the pcDNA3gagml/pcDNA3gagpolfusionml combination was less than half that obtained with pcDNA3gagpolml. One possible explanation for this discrepancy is that translation of the two polyproteins from the same RNA results in their co-localisation thereby facilitating virion assembly. It is interesting to note that Wu et al. (2000) also report a three- to five-fold decrease in titre when Gag and Pol were expressed separately using a somewhat different approach.

Western blot analysis using anti HIV-1 serum showed no apparent differences in the processing of p24 made from pcDNA3gagpolml or pcDNA3gag/pcDNA3gagpolfusionml in comparison to pCMVΔRnr demonstrating that neither codon-optimisation, nor the physical separation of the Gag and GagPol reading frames, has a significant effect on processing of Gag.

The expression of Gag and GagPol from separate reading frames has several important consequences. Firstly, it allows the removal of a vital HIV-1 cis element, the Gag/GagPol translational frameshift signal from the virus production system. Secondly, as neither sequence can support the production of virus particles it greatly reduces the probability of the constituents of the system recombining to generate replication-competent virus. Indeed it is difficult to see how recombination events alone could lead to the generation of replication-competent virus from the constituents of our virus production system when separate constructs are used for expression of the Gag and GagPol polyproteins.

Safety Assessment

Wu et al. (2000) have described a somewhat similar system where Gag-Pro and Vpr-RT-IN polyproteins are expressed separately. In this instance RT-IN is expressed as a fusion protein with Vpr to facilitate its incorporation into the virion. However, they have not used separate plasmids for the expression of Tat, Rev or Vif (the only minor protein expressed by their system) and retain the frameshift signal for expression of Pro. It should be noted that there is a region of overlap between their Gag-Pro and RT-IN constructs although the RT and IN reading frames are blocked by stop codons in the Gag-Pro expression construct.

In our view the demonstration of the efficacy of our approach, as well as those of others, depends on the development of systems for the unitary detection of replication-competent virus and/or precursors to replication-competent virus. It is important that such assays for replication-competent virus make as few assumptions as possible about the phenotype of the virus. In this regard assays for HIV-1 replication are probably unsuitable. We have analysed batches of virus produced with both our helper plasmids, and with pCMVΔRnr, for transfer of Gag expression (assayed by p24 ELISA) to transduced cells with consistently negative results (data not shown). This suggests that this approach is inadequate for comparative assessment of the safety of the different virus production systems. We therefore developed assays designed to detect transfer to transduced cells of biologically active sequences encoding either the Gag polyprotein alone (gag transfer assay), or sequences the equivalent of the gagpol gene (gagpol transfer assay). The results of these assays demonstrate that the use of a codon-optimised reading frame decreases, but does not completely abolish, the transfer of such sequences. This is not surprising as the codon-optimised reading frames still have short stretches of absolute sequence homology with the native gagpol gene sequence that will allow homologous recombination between the vector gag sequence and the codon-optimised helper sequence. Others (Wagner et al., 2000) have also shown that codon-optimisation improves safety but in this instance an assay for replication-competent virus was used and no readout detected with virus made using their codon-optimised construct. This suggests that our assays provide a more stringent assessment of safety (at least in terms of measuring transfer of sequences expressing HIV-1 Gag/GagPol polyproteins) and that it is therefore a more appropriate assay to use for comparative and absolute assessment of different HIV-1 packaging systems. With our split helper system utilising separate plasmids for expression of the Gag and GagPol polyproteins we have not been able to detect the coincident transfer of sequences capable of expressing Gag and GagPol (i.e capable of substituting for the gagpol gene) using the gagpol transfer assay. However, it still remains to be determined whether our assay is capable of detecting single events (i.e. transfer of a single intact reading frame) or has a higher threshold of detection.

Secondary Proteins

With the exception of Vpr we were unable to demonstrate expression of the HIV-1 minor proteins. This may be due to the fact that the antibodies used for analysis were raised to the corresponding proteins from the HIV-1 HXB2 strain and also displayed a high level of cross reactivity with cellular proteins. Due to the simplicity of our constructs we assume that our constructs are expressing these proteins. Expression of HIV-1 BH10 Nef from pcDNA3.1 has been described by others (Cooke et al., 1997) supporting this view. Our results show that, in combination, the minor proteins cause a small but significant increase in virus titre and more notably, appear to increase the efficiency of packaging (i.e. titre per ng of p24). There is published evidence that both supports and contradicts this finding. For example, the HIV-1 accessory proteins are required for the transduction of quiescent lymphocytes with HIV-1 based vectors (Chinnasamy et al., 2000). Additionally, although Zufferey et al. (1997) reported that removal of the minor proteins from packaging systems has no detrimental effect on virus titre, Srinivasakumar and Schuening (1999) reported that Vpr and the combination of Vpr and Vpu increased virus particle production. To this end it will be interesting to see what phenotype results from the use of a VprRT-IN fusion by Wu et al. (2000) and if this approach allows the subsequent addition of Vpr to their system.

Regulatory Proteins

Analysis of the requirement for the regulatory proteins Rev and Tat shows that despite our ability to express high levels of the HIV-1 structural proteins without the inclusion of the RRE in the transcriptional unit, Rev is absolutely necessary for efficient virus production. This is presumably due to the dependence of our vector on the Rev/RRE system for efficient packaging. To date we have not been able to construct a Rev-independent vector with the use of heterologous constitutive nuclear export signals to replace the Rev/RRE function (manuscript in preparation). However, if possible, this would, in combination with our codon-optimised gagpol sequence, allow the generation of a completely Rev-independent vector system.

Tat is also required for maximal production of virus. This is expected as production of genomic transcripts from our vector construct is mediated by the HIV-1 LTR (FIG. 12). However, the effect of Tat on virus titre is much less than the level of trans-activation of transcription from the viral LTR by Tat (which is at least 50-fold). This disparity may simply reflect the conditions under which the experiment is done, if other factors in virus assembly are limiting differences in vector RNA expression will be of less consequence in determining virus titre. It has also been suggested that Tat has a role in the reverse transcription stage of the HIV-1 life cycle (Harrich et al., 1997) although, if anything, this would be expected to increase the requirement for Tat.

High Multiplicity of Infection

To determine whether the lower titres per ng of p24 obtained with our multipartite systems, compared to those obtained with pCMVΔRnr, resulted in lower transduction at a high moi (as a model for the likely in vivo situation), A549 cells were transduced at a moi of 0.1, 1 or 10 with virus prepared with pCMVΔRnr or pcDNA3gagpolml. Although the latter virus had a titre/ng p24 only 40% of the former it had only a slightly lower ability to transduce cells in terms of the number of positive cells resulting from transduction. This was most notable at a moi of 1 where transduction with the pcDNA3gagpolml virus was only 78% of that obtained with the pCMVΔRnr virus. At an moi of 10, both viruses resulted in greater than 95% transduction showing there is no absolute limitation on achieving high levels of transduction with the virus with the lower titre/ng p24. However, analysis of the mean fluorescence of the positive cells suggests that the effect is more pronounced. At an moi of 10 the mean fluorescence of the cells transduced with the pcDNA3gagpolml virus is over 3 fold lower than for the cells transduced with the pCMVΔRnr virus. We believe that optimisation of our various expression constructs and of the vector used, as well as the protocols used for virus production, will help address this problem. Also of concern is that we have noted that high-titre stocks, which because of the lower titres produced with our systems require higher degrees of (volume) concentration for their production, can result in noticeable toxicity on some cell types after prolonged exposure. This may indicate that more refined virus purification procedures may be required for the preparation of high-titre stocks for some applications.

TABLE 1
Expression of gagpol gene products from
single transcriptional unit constructs.

| Construct | p24 | RT | Titre |
|---|---|---|---|
| (ng/transfection) | ng/ml | ng/ml | EYFP TU/ml |
| pcDNA3gagpolml (653) | 477 ± 35 | 3.26 ± 0.90 | 2.4 ± 0.3 × 10⁵ |
| pcDNA3gagpolmlrre (671) | 392 ± 12 | n.d. | 1.9 ± 0.01 × 10⁵ |
| pCMVΔRnr (850) | 597 ± 103 | 2.22 ± 0.94 | 6.6 ± 1.0 × 10⁵ |

n = 4 for all values;
n.d. not determined.
For each experiment an equivalent molar amount (100 fmoles) of the relevant GagPol expression plasmid (ng quantity of plasmid given in brackets) was co-transfected with 1 μg of pBlHIVext5SV40EYFPppt + RRELTR, 0.5 μg of pHCMV-G, 0.5 μg of pcDNA3Tat and 0.5 μg of pCMV-rev.

TABLE 2
Expression of Gag and GagPol polyproteins
from separate transcriptional units 1.

| | p24 | RT | Titre |
|---|---|---|---|
| Plasmids | ng/ml | ng/ml | EYFP TU/ml |
| pcDNA3gagml | 96 ± 9 | n.d. | n.d. |
| pcDNA3gagml: | 567 ± 22 | 2.45 | 1.2 ± 0.1 × 10⁵ |
| pcDNA3gagpolfusionml | | (2.91, 1.99) | |
| pcDNA3gagmlrre: | 572 ± 12 | ND | 1.3 ± 0.1 × 10⁵ |
| pcDNA3gagpolfusionmlrr | | | |
| e | | | |
| pcDNA3gagmlcte: | 643 ± 46 | ND | 1.3 ± 0.2 × 10⁵ |
| pcDNA3gagpolfusionmlct | | | |
| e | | | |
| pcDNA3gagpolfusionml | 87 ± 26 | 2.10 | n.d. |

n = 3 for all p24 and titre results, n = 2 for RT result pcDNA3gagml and n = 1 for pcDNA3gagpolfusionml;
n.d. not detected,
ND not determined.
For each experiment 100 fmoles of the Gag plasmid (455 ng of pcDNA3gagml, 462 ng of pcDNA3gagmlrre and pcDNA3gagmlcte) were transfected with 20 fmoles of GagPolfusion (131 ng of pcDNA3gagpolfusionml, 132 ng of pcDNA3gagpolfusionmlrre and pcDNA3gagpolfusionmlcte) and 1 μg of pBlHIVext5SV40EYFPppt + RRELTR, 0.5 μg of pHCMV-G, 0.5 μg of pcDNA3Tat and 0.5 μg of pCMV-rev.

TABLE 3
Expression of Gag and Pol
from separate transcriptional units.

| Molar ratio of | | | |
|---|---|---|---|
| pcDNA3gagml: | p24 | RT | Titre |
| pcDNA3polml | ng/ml | ng/ml | EYFP TU/ml |
| 1:0 | 568 ± 41 | n.d. | n.d. |
| 10:1 | 1205 ± 95 | 23 ± 7 | n.d. |
| 4:1 | 1376 ± 74 | 118 ± 4 | n.d. |
| 1:1 | 1573 ± 116 | 283 ± 38 | n.d. |
| 0:1 | n.d. | 316 ± 20 | n.d. |

n = 3 for all results;
n.d., not detected.
For each experiment 455 ng of pcDNA3gagml (100 fmoles) were transfected with the appropriate amount of pcDNA3polml (56, 140 or 560 ng, 10 to 100 fmoles) and 1 μg of pB1HIVext5SV40EYFPppt + RRELTR, 0.5 μg of pHCMV-G, 0.5 μg of pcDNA3Tat and 0.5 μg of pCMV-rev.

TABLE 4
Effect of Tat expression on virus titre.

| | Titre EYFP | p24 | Titre EYFP |
|---|---|---|---|
| Construct | TU/ml | ng/ml | TU /ng p24 |
| “Complete mix” | 1.9 ± 0.6 × 10⁵ | 473 ± 145 | 409 ± 78 |
| w/o pcDNA3Tat | 0.7 ± 0.2 × 10⁵ | 299 ± 110 | 246 ± 44 |

n = 9 for all experiments;
n.d. not detected.
“Complete mix” contains pcDNA3gagpolml (100 fmoles, 653 ng), pB1HIVext5SV40EYFPppt + RRELTR (1 μg), pHCMV-G (0.5 μg), pcDNA3Tat (0.5 μg) and pCMV-rev (0.5 μg). “W/o Tat” contains pcDNA3gagpolml (100 fmoles, 653 ng), pB1HIVext5SV40EYFPppt + RRELTR (1 μg), pHCMV-G (0.5 μg) and pCMV-rev (0.5 μg).

TABLE 5
Effect of Rev expression on virus titre.

| | Titre EYFP | p24 | Titre EYFP |
|---|---|---|---|
| Construct | TU/ml | ng/ml | TU/ng p24 |
| “Complete mix” | 1.9 ± 0.6 × 10⁵ | 473 ± 145 | 409 ± 78 |
| w/o pCMV-rev | n.d | 287. | n.d. |

“Complete mix” (n = 9) contains pcDNA3gagpolml (100 fmoles, 653 ng), pB1HIVext5SV40EYFPppt + RRELTR (1 μg), pHCMV-G (0.5 μg), pcDNA3Tat (0.5 μg) and pCMV-rev (0.5 μg). “W/o pCMV-rev”, (n = 1) contains pcDNA3gagpolml (100 fmoles, 653 ng), pB1HIVext5SV40EYFPppt + RRELTR (1 μg), pHCMV-G (0.5 μg) and pcDNA3Tat (0.5 μg).
n.d. not detected.

TABLE 6
Effect of minor proteins on virus titre.

| | Titre EYFP | p24 | Titre EYFP |
|---|---|---|---|
| Construct | TU/ml | ng/ml | TU/ng p24 |
| w/o minor proteins | 1.5 ± 0.2 × 10⁵ | 321 ± 36 | 477 ± 80 |
| plus minor proteins | 1.9 ± 0.4 × 10⁵ | 211 ± 28 | 895 ± 150 |

n = 9 for both experiments.
pB1HIVext5SV40EYFPppt + RRELTR (0.4 μg), pcDNA3gagpolml (0.4 μg), pcDNA3Tat (0.4 μg), pCMV-rev (0.4 μg) and pCMV-G (0.2 μg) were co-transfected as described in Materials and methods, either with or without all 4 minor protein (pcDNA3Vpu/Vpr/Vif/Nef) expression constructs (0.3 μg of each).

TABLE 7
Transfer of gag and gagpol
sequences via recombinant virus stocks.

| | gag assay | gagpol assay |
|---|---|---|
| | Neo^{r} colonies/10⁶ | Neo^{r} colonies/10⁶ |
| GagPol helper construct(s) | EYFP TU | EYFP TU |
| pCMVΔRnr | 619 ± 140 | 1837 ± 709 |
| pcDNA3gagpolml | 7 ± 5 | 21 ± 11 |
| pcDNA3gagml/ | 48 ± 42 | 0.7 ± 0.5 |
| pcDNA3gagpolfusionml | | |
| 293T control* | 0.3 ± 0.5 | 0.7 ± 0.5 |

Samples of virus prepared with either the pCMVΔRnr, pcDNA3gagpolml or the pcDNA3gagml/pcDNA3gagpolfusionml packaging systems were used to transduce 293T cells which were then assayed for expression of biologically active Gag and Gag/GagPol coding sequences as described in Materials and methods.
*Absolute number of colonies given as no virus input.
n = 3 for all results.

TABLE 8
Transduction of NIH3T3 cells at high moi.

| | % transduction/mean F1* | | |
|---|---|---|---|
| | M1 | | |
| Helper construct (titre/ng p24) | moi 0.1 | moi 1.0 | moi 10 |
| pCMVΔRnr (4516 TU/ng) | 13/152 | 67/276 | 99/158 |
| | | | 2 |
| pcDNA3gagpolml (1876 | 11/144 | 52/213 | 96/470 |
| TU/ng) | | | |

A549 cells were transduced in duplicate at an moi of approximately 0.1, 1 and 10 using virus prepared with either the pCMVΔRnr or the pcDNA3gagpolml packaging systems and the pB1HIVext5SV40EYFPppt+RRELTR vector. Three days post transduction, cells were assayed by FACSCAN analysis. The results are given as percentage EYFP positive cells and the mean fluorescence of those positive cells (M1).

### REFERENCES

Albelda et al., Annals of Internal Medicine. 132, 649 (2000)

Amado and Chen (1999). Science July 30 285, 674-676.

Anson and Fuller (2001) Hum Gene Ther 12, 2085

Aurichio et al., (2002) J. Clin. Invest. 110:499-504

Ausubel et al., (1989). Current Protocols in Molecular Biology. New York: Wiley Intersciences.

Bergelson et al., (1997). Science 275, 1320-1323.

Besterman and Domanico (1992). Biochemistry 31, 2046-2056.

Bevins (1999). American Journal of Respiratory Cell & Molecular Biology 20, 861-863.

Blomer et al., (1997). J. Virol. 71, 6641-6649.

Borthwick et al., (2001). American Journal of Respiratory Cell & Molecular Biology 24, 662-670.

Chang et al., (1999). Gene Ther. 6, 715-728.

Chinnasamy et al., (2000). Blood 96, 1309-1316.

Chu et al., (2001). Human Gene Therapy 12, 455-467.

Cipolla et al., (2000) Hum Gene Ther 11, 361-371

Coelen et al., (1983). Arch Virol 75, 307-311.

Cooke et al., (1997). J. Gen. Virol. 78, 381-392.

Cosset et al., (1995). J. Virol. 69, 7430-7436.

Coyne et al., (2000). American Journal of Respiratory Cell & Molecular Biology 23, 602-609.

Cui et al., (1999). J. Virol. 73, 6171-6176.

Davies et al., (2001). Current Opinion in Pharmacology 1, 272-278.

Duan et al., (1998). Hum Gene Ther 9, 2761-2776.

Dull et al., (1998). J. Virol. 72, 8463-8471.

Engelhardt (2001). Am J Respir Cell Mol Biol 24,649-652.

Friedman. (2000). Science 287, 2163-2165.

Fuller and Anson. (2001). Human Gene Therapy 12, 2081-2093.

Harrich et al., (1997). EMBO J 16, 1224-1235.

Hodson and Geddes (1995). Cystic Fibrosis. (Chapman & Hall Medical, London).

Iwakuma et al., (1999). Virol. 261, 120-132.

Johnson et al., (2000). Gene Therapy 7, 568-574.

Johnson et al., (1992). Nature Genetics 2, 21-25.

Jordan et al., N.A.R. 4, 596 (1996)

Jospeh et al., (2001) Hum Gene Ther 12, 1369-1382

Kafri et al, (1997). Nat. Genet. 17, 314-317.

Kay et al., Nature Medicine. 7, 33 (2001)

Kim et al., (1998). J. Virol. 72, 811-816.

Klages et al., (2000) Mol. Ther. 2, 170-176.

Knowles et al., (1995) Hum Gene Ther 6, 445-455

Knowles and Boucher (2002). J Clin Invest 109, 571-577.

Kobinger et al., (2001). Nature Biotechnology 19, 225-230.

Koehler et al., (2001). Molecular Therapy: the Journal of the American Society of Gene Therapy 4, 84-91.

Korin and Zack. (1998). J. Virol. 72, 3161-3168.

Kotsopoulou et al., (2000). J. Virol. 74, 4839-4852.

Lee et al., (1999) Hum Gene Ther 10, 603-613

Lewis et al., (1990). J. Virol. 64, 1690-1697.

Lewis et al, (1992). EMBO J. 11, 3053-3058.

Li et al., (1992). J. Virol. 66, 6587-6600.

Martin et al., (1978). Gut 19, 103-107.

Merkus et al., (1993). Journal of Controlled Release 24, 201-208.

Mery et al., (1994). Toxicologic Pathology 22, 353-372.

Middleton et al., (1994) Eur Respir 7:2050-2056

Miyoshi et al., (1997) P.N.A.S. (USA) 94, 10319-10323.

Miyosh et al., (1998). J. Virol. 72, 8150-8157.

Mulligan, Science. 260, 926 (1993)

Naldini et al., (1996). Science 272, 263-267.

Niewoehner et al., (1989). Journal of Applied Physiology 66, 261-267.

Park et al., (2000). Nat. Genet. 24, 49-52.

Parsons et al., (2000a). Gene Therapy 7, 1810-1815.

Parsons et al., (2000b). Respirology 5, A52 (Abstract).

Parsons et al., (1999). Respirology 4, A11 (Abstract).

Parsons et al., (1998). Human Gene Therapy 9, 2661-2672.

Pasquinelli et al., (1997). EMBO J. 16, 7500-7510.

Pickles et al., (2000). Journal of Virology 74, 6050-6057.

Poeschla et al., (1998). Nat. Med. 4, 354-357.

Prokazova et al., (1998). Biochemistry (Moscow) 63, 31-37.

Rhee and Hunter (1991). EMBO J. 10, 535-546.

Rigg et al., (1996). Virol. 218, 290-295.

Sadaie et al., (1998). J. Med. Virol. 54, 118-128.

Salzman et al., (1985) New England Journal of Medicine 312,1078-1084

Seidner et al., (1988). Biochimica et Biophysica Acta 961, 328-336.

Shioda and Shibuta. (1990). Virol. 175, 139-148.

Siegfried et al., (1995) Am. J. Respir. Cell. Mol. Biol. 12,379-384

Snouwaert et al., (1992). Science 257, 1083-1088.

Srinivasakumar and Schuening (1999). J. Virol. 73, 9589-9598.

Stemmer et al., (1995). Gene 164, 49-53.

Sutton et al., (1998). J. Virol. 72, 5781-5788.

Sutton et al., (1999). J. Virol. 73, 3649-3660.

Tarran et al., (2001). Molecular Cell 8, 149-158.

Wagner et al.,. (2000). Hum. Gene Ther. 11, 2403-2413.

Wand et al., (2000). American Journal of Respiratory Cell & Molecular Biology 22, 129-138.

Weltzein. (1979). Biochimica et Biophysica Acta 559, 259-287.

Wu et al.,. (2000). Mol. Ther. 2, 47-55.

Xu et al.,. (2001). Mol. Ther. 3, 97-104.

Yee et al., (1994). Proc. Natl. Acad. Sci. USA 91, 9564-9568.

Zufferey et al., (1997). Nat. Biotech. 15, 871-875.

1. A method of delivering one or more exogenous genes for expression in an epithelial cell in the respiratory system of a mammal to give presistent expression of the gene in the epithelial cell, the method including the steps of delivering an effective amount of a penetrating agent to cause tolerable transient damage to superficial epithelial cells of the respiratory system, and the step of delivering a recombinant exogenous gene in a lentiviral particle, the lentiviral particle containing a non-replicating nucleic acid, the nucleic acid encoding the exogenous gene operable linked to a control sequence for controlling expression of the gene.

2. A method of delivering one or more exogenous genes for expression in an epithelial cell in the respiratory system of a mammal as in claim 1 wherein the penetrating agent is a detergent.

3. A method of delivering one or more exogenous genes for expression in an epithelial cell in the respiratory system of a mammal as in claim 2 wherein the detergent is selected from one or more of the classes of detergent consisting of the group, anionic detergent, zwitterionic detergent, ionic detergent.

4. A method of delivering one or more exogenous genes for expression in an epithelial cell in the respiratory system of a mammal as in claim 2 wherein the detergent is selected from the group consisting of, Sodium n-dodecyl sulfate, Zwittergent 3-14: N-Tetradecylsulfobetaine, Cetyltrimethylammonium bromide, Deoxycholic Acid, lysophophatidylcholine (LPC) and polidocanol.

5. A method of delivering one or more exogenous genes for expression in an epithelial cell in the respiratory system of a mammal as in claim 2 wherein the detergent is added at concentrations of between 0.005 and 0.5% v/v.

6. A method of delivering one or more exogenous genes for expression in an epithelial cell in the respiratory system of a mammal as in claim 2 wherein the detergent is lysophophatidylcholine.

7. A method of delivering one or more exogenous genes for expression in an epithelial cell in the respiratory system of a mammal as in claim 6 wherein LPC is delivered at concentrations of 0.01 to 3% v/v.

8. A method of delivering one or more exogenous genes for expression in an epithelial cell in the respiratory system of a mammal as in claim 6 wherein LPC is delivered at concentrations of 0.05 to 1% v/v.

9. A method of delivering one or more exogenous genes for expression in an epithelial cell in the respiratory system of a mammal as in claim 6 wherein LPC is delivered at concentrations of about 0.1% v/v.

10. A method of delivering one or more exogenous genes for expression in an epithelial cell in the respiratory system of a mammal as in claim 7 wherein delivery of LPC and lentivirus is not concurrent.

11. A method of delivering one or more exogenous genes for expression in an epithelial cell in the respiratory system of a mammal as in claim 10 wherein the LPC is delivered before the lentivirus by a time of 12 hours or less.

12. A method of delivering one or more exogenous genes for expression in an epithelial cell in the respiratory system of a mammal as in claim 10 wherein the LPC is delivered before the lentivirus by a time of about 1 hour.

13. A method of delivering one or more exogenous genes for expression in an epithelial cell in the respiratory system of a mammal as in claim 1 wherein persistence is greater than 46 days.

14. A method of delivering one or more exogenous genes for expression in an epithelial cell in the respiratory system of a mammal as in claim 1 wherein persistence is greater than 92 days.

15. A method of delivering one or more exogenous genes for expression in an epithelial cell in the respiratory system of a mammal as in claim 1 wherein persistence is greater than 13 months.

16. A method of delivering one or more exogenous genes for expression in an epithelial cell in the respiratory system of a mammal as in claim 1 wherein the exogenous gene is expressed in sufficient numbers of cells and amounts to provide an ameliorating effect for a respiratory condition.

17. A method of delivering one or more exogenous genes for expression in an epithelial cell in the respiratory system of a mammal as in claim 16 wherein the exogenous gene is CFTR and the condition is cystic fibrosis.

18. A method of delivering one or more exogenous genes for expression in an epithelial cell in the respiratory system of a mammal as in claim 17 wherein the expression of the CFTR gene is sufficient to provide a significant shift of a reduced ΔPD back to normal levels in the mammal.

19. A method of delivering one or more exogenous genes for expression in an epithelial cell in the respiratory system of a mammal as in claim 17 wherein the persistence is 46 days or more.

20. A method of delivering one or more exogenous genes for expression in an epithelial cell in the respiratory system of a mammal as in claim 18 wherein the persistence is 46 days or more.

21. A method of delivering one or more exogenous genes for expression in an epithelial cell in the respiratory system of a mammal as in claim 1 wherein the cell is non-terminally differentiated.

22. A method of delivering one or more exogenous genes for expression in an epithelial cell in the respiratory system of a mammal as in claim 21 wherein cell is capable of differentiating into two or more of cell classes consisting of the group comprising ciliated cells, non-ciliated cells, secretory cells and basal cells.

23. A method of delivering one or more exogenous genes for expression in an epithelial cell in the respiratory system of a mammal as in claim 21 wherein exogenous gene is enzymic.

24 A method of delivering one or more exogenous genes for expression in an epithelial cell in the respiratory system of a mammal as in claim 1 wherein the tolerable transient damage is such that transepithelial potential difference is disrupted by the delivery of the penetrating agent and is returned to normal or near normal within about 2 days.

25. A method of delivering one or more exogenous genes for expression in an epithelial cell in the respiratory system of a mammal as in claim 24 wherein the transepithelial potential difference is returned to normal or near normal within about 1 day.

26 A method of delivering one or more exogenous genes for expression in an epithelial cell in the respiratory system of a mammal as in claim 24 wherein the transepithelial potential difference is returned to normal or near normal within about 6 hours.

27. A recombinant lentiviral packaging system, including a first nucleic acid molecule including a gag gene sequence encoding a Gag protein, and a second nucleic acid molecule including a gagpol gene sequence encoding a GagPol protein, the gagpol gene sequence having a degenerative nucleotide changes in the frame shift sequence AUUUUUU to reduce the chance of a frameshift which switches expression of the Gagpol protein to the Gag protein in wild type lentivirus, the packaging system additionally including a lentiviral vector nucleic acid molecule not encoding either the gag gene or the gagpol gene or both.

28 A recombinant lentiviral packaging system as in claim 27 wherein the degenerative nucleotide change in the gagpol gene sequence is such that the gagpol gene can no longer encode the gag gene on the frame shift mutation.

29. A recombinant lentiviral packaging system as in claim 27 wherein the degenerative nucleotide change in the gagpol gene sequence is to the sequence ACUUCCU.

30 A recombinant lentiviral packaging system as in claim 27 wherein the gagpol gene sequence has additionally degenerate nucleotide substitutions which destabilise the hairpin structure associated with the frameshift event.

31. A recombinant lentiviral packaging system as in claim 27 wherein the gag gene is a truncation of the wild type gagpol gene so that it can no longer be translated to form Gagpol.

32. A recombinant lentiviral packaging system as in claim 27 wherein the lentivirus is HIV or HIV derived.

33. A recombinant nucleic acid molecule encoding a lentiviral gagpol gene having a degenerative nucleotide changes in the frame shift sequence AUUUUUU to reduce the chance of a frameshift which switches expression of the Gagpol protein to the Gag protein in wild type lentivirus.

34. A recombinant nucleic acid molecule as in claim 33 wherein the degenerative nucleotide change in the gagpol gene sequence is to the sequence ACUUCCU.

35 A recombinant nucleic acid molecule as in claim 33 wherein the gagpol gene sequence has additionally degenerate nucleotide substitutions which destabilise the hairpin structure associated with the frameshift event.

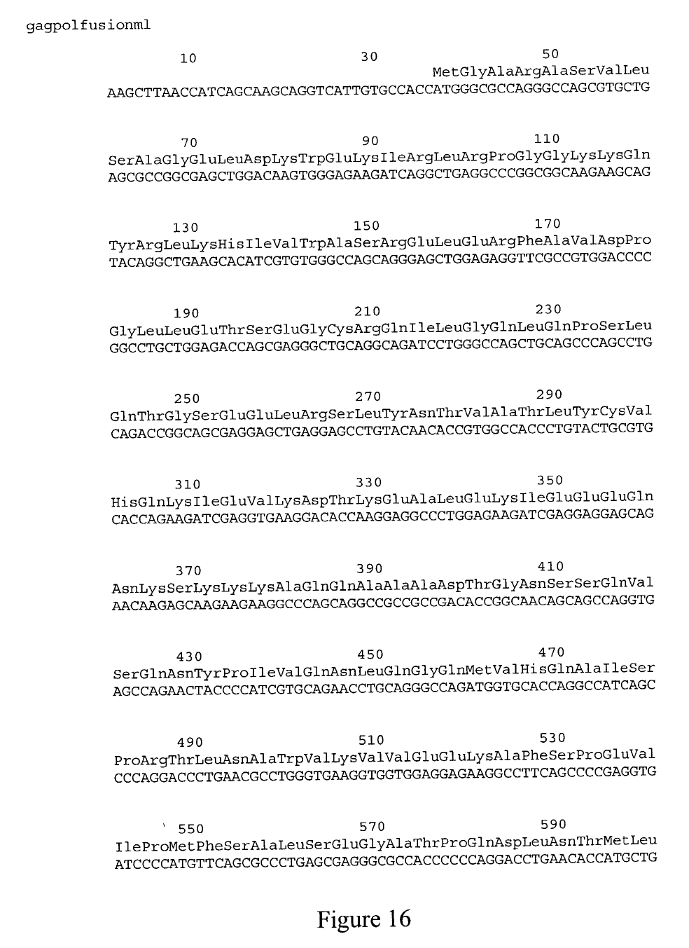

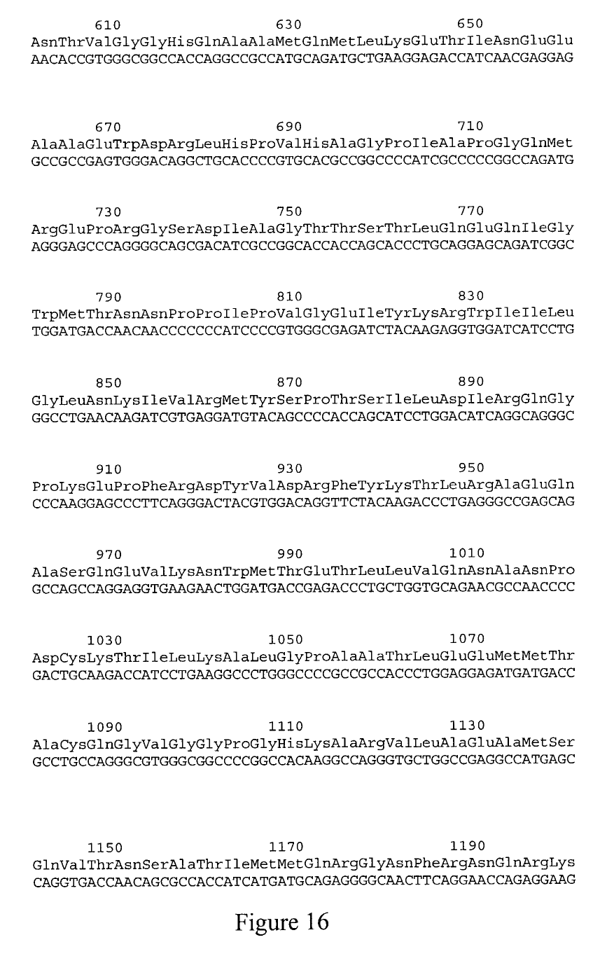

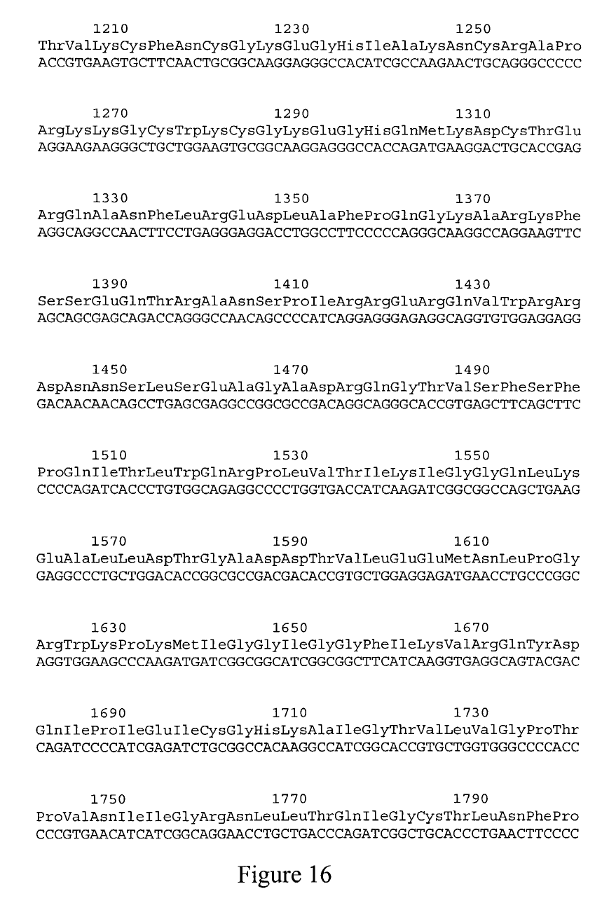

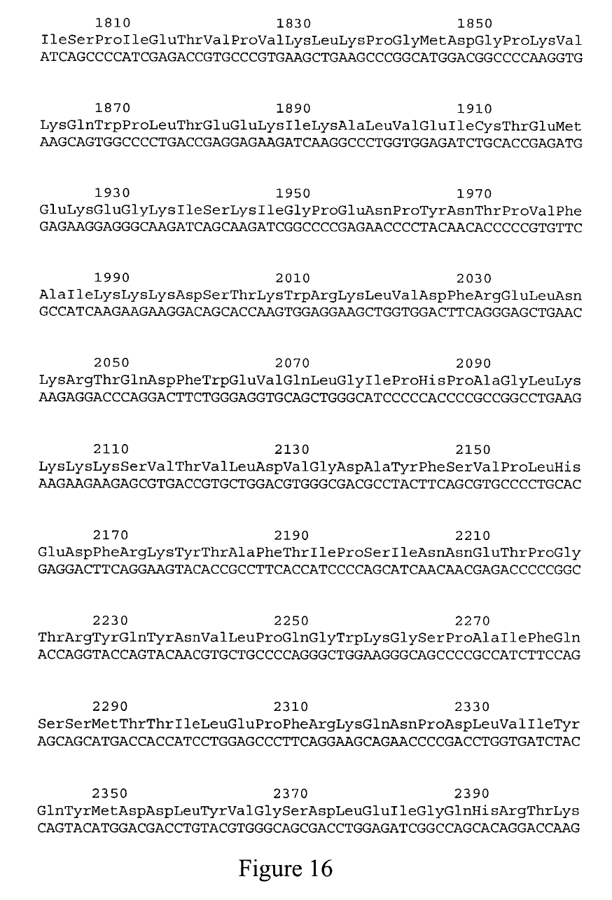

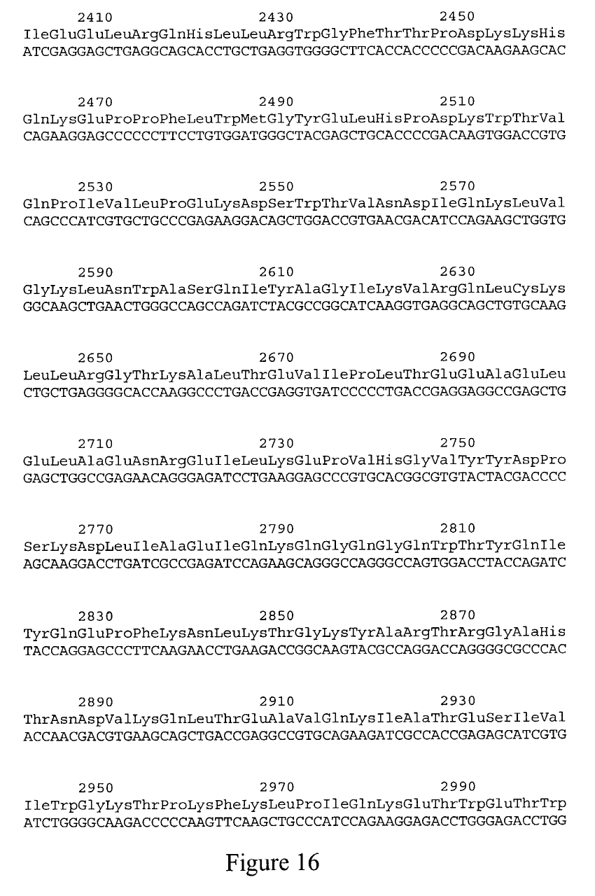

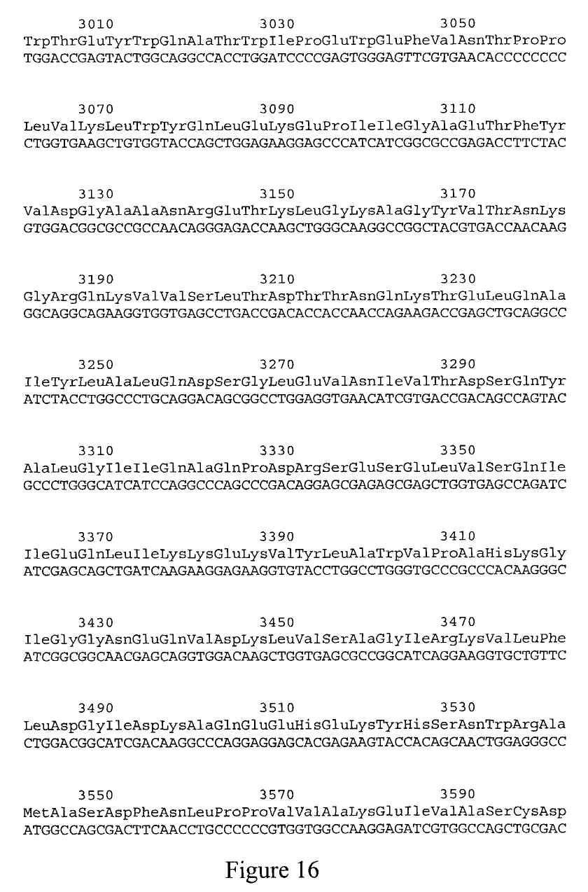

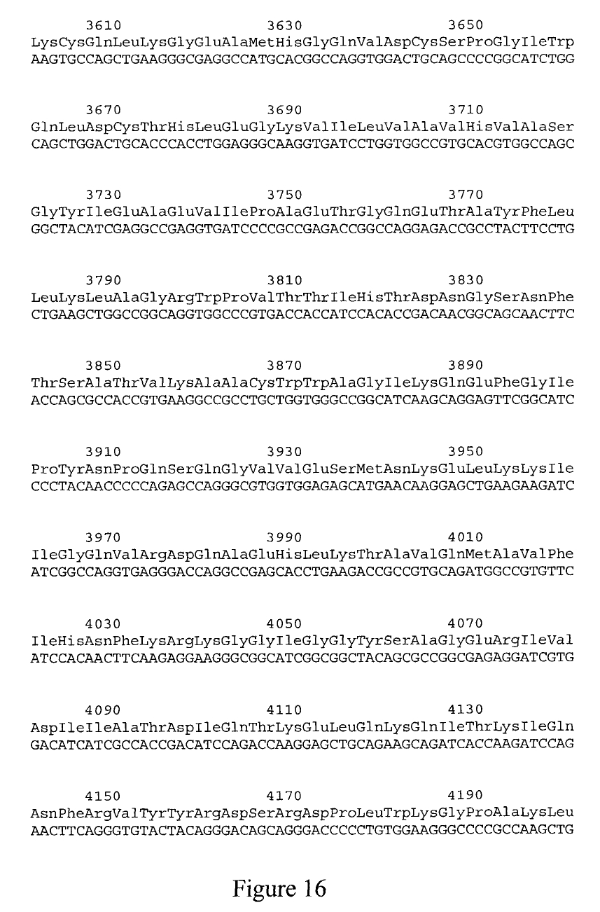

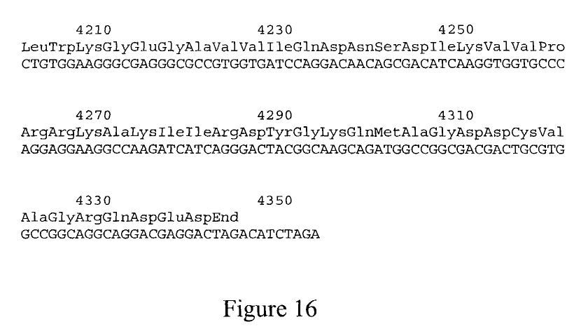

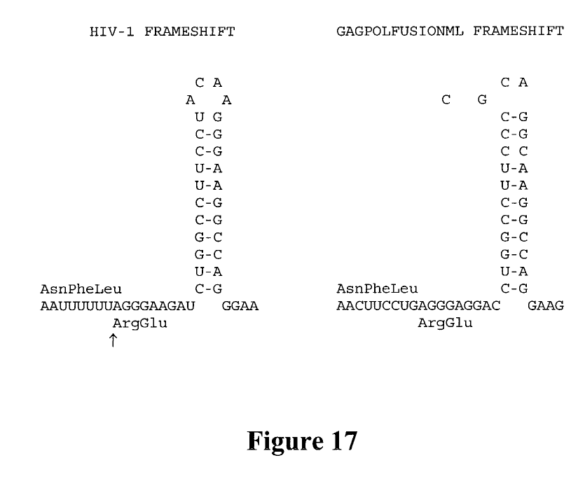

36. A recombinant nucleic acid molecule as in claim 33 wherein the molecule is that set out in FIG. 16.
